SEQUENCE LISTING (38 sequences)

SEQ ID NO: 1 - protein, 6 aa, Artificial Sequence
  Linker peptide.
LGSGGH

SEQ ID NO: 2 - DNA, 27 bp, Artificial Sequence
  Cloning linker.
aatttgggatccggcggtcatatgact

SEQ ID NO: 3 - DNA, 88 bp, Artificial Sequence
  Cloning sequence.
tatgcaccaccaccaccaccacgatatcgccaaacttaaggccggcgctagcttgggatccggcggtcatatgactagtgccaaaaag

SEQ ID NO: 4 - DNA, 90 bp, Artificial Sequence
  Cloning sequence.
aattctttttggcactagtcatatgaccgccggatcccaagctagcgccggccttaagtttggcgatatcgtggtggtggtggtggtgca

SEQ ID NO: 5 - DNA, 40 bp, Artificial Sequence
  PCR primer.
aaaaaagatatcgagaaaaaaatcactggatataccaccg

SEQ ID NO: 6 - DNA, 30 bp, Artificial Sequence
  PCR primer.
aaaaaagctagccgccccgccctgccactc

SEQ ID NO: 7 - DNA, 43 bp, Artificial Sequence
  PCR primer.
aaaaaacttaagactgaaaaaaaatatatcgttgcgctcgacc

SEQ ID NO: 8 - DNA, 34 bp, Artificial Sequence
  PCR primer.
aaaaaagctagcttcgtcgtgttcttcccacgcc

SEQ ID NO: 9 - DNA, 31 bp, Artificial Sequence
  DNA ligand.
ctttagttacaacatacttatccccgccccc

SEQ ID NO: 10 - DNA, 31 bp, Artificial Sequence
  DNA ligand.
gaaatcaatgttgtatgaataggggcggggg

SEQ ID NO: 11 - DNA, 33 bp, Artificial Sequence
  DNA ligand.
ctttagttacaacatacttatcaccccgccccc

SEQ ID NO: 12 - DNA, 31 bp, Artificial Sequence
  DNA ligand.
aatcaatgttgtatgaatagtggggcggggg

SEQ ID NO: 13 - DNA, 33 bp, Artificial Sequence; identical to SEQ ID NO: 11
  DNA ligand.

SEQ ID NO: 14 - DNA, 30 bp, Artificial Sequence
  DNA ligand.
atcaatgttgtatgaatagtggggcggggg

SEQ ID NO: 15 - DNA, 36 bp, Artificial Sequence
  DNA ligand.
cccccgcccccaatactttagttacaacatacttat

SEQ ID NO: 16 - DNA, 31 bp, Artificial Sequence
  DNA ligand.
gggggcgggggttatgaaatcaatgttgtat

SEQ ID NO: 17 - DNA, 33 bp, Artificial Sequence
  DNA ligand.
aattagtatgttgtaactaaagtgggggcgggg

SEQ ID NO: 18 - DNA, 33 bp, Artificial Sequence
  DNA ligand.
ttaatcatacaacattgatttcacccccgcccc

SEQ ID NO: 19 - DNA, 33 bp, Artificial Sequence
  DNA ligand.
aataagtatgttgtaactaaagtgggggcgggg

SEQ ID NO: 20 - DNA, 33 bp, Artificial Sequence
  DNA ligand.
ttattcatacaacattgatttcacccccgcccc

SEQ ID NO: 21 - DNA, 33 bp, Artificial Sequence
  DNA ligand.
atataggatgttgtaactaatatgggggcgggg

SEQ ID NO: 22 - DNA, 33 bp, Artificial Sequence
  DNA ligand.
tatatcctacaacattgattatacccccgcccc

SEQ ID NO: 23 - DNA, 33 bp, Artificial Sequence
  DNA ligand.
cattagtatgttgtaactaaatggggggcgggg

SEQ ID NO: 24 - DNA, 33 bp, Artificial Sequence
  DNA ligand.
gtaatcatacaacattgatttaccccccgcccc

SEQ ID NO: 25 - DNA, 33 bp, Artificial Sequence
  DNA ligand.
ttaaagtatgttgtaactaagcagggggcgggg

SEQ ID NO: 26 - DNA, 33 bp, Artificial Sequence
  DNA ligand.
aatttcatacaacattgattcgtcccccgcccc

SEQ ID NO: 27 - DNA, 33 bp, Artificial Sequence
  DNA ligand.
ccttcgtatgttgtaacgacgatgggggcgggg

SEQ ID NO: 28 - DNA, 33 bp, Artificial Sequence
  DNA ligand.
ggaagcatacaacattgctgctacccccgcccc

SEQ ID NO: 29 - DNA, 33 bp, Artificial Sequence
  DNA ligand.
gtcaaggatgttgtaactaaccagggggcgggg

SEQ ID NO: 30 - DNA, 33 bp, Artificial Sequence
  DNA ligand.
cagttcctacaacattgattggtcccccgcccc

SEQ ID NO: 31 - DNA, 33 bp, Artificial Sequence
  DNA ligand.
cgatcgtatgttgtaactatctcgggggcgggg

SEQ ID NO: 32 - DNA, 33 bp, Artificial Sequence
  DNA ligand.
gctagcatacaacattgatagagcccccgcccc

SEQ ID NO: 33 - DNA, 33 bp, Artificial Sequence
  DNA ligand.
aacatggaagttgtaactaaccggggggcgggg

SEQ ID NO: 34 - DNA, 33 bp, Artificial Sequence
  DNA ligand.
ttgtaccttcaacattgattggccccccgcccc

SEQ ID NO: 35 - DNA, 33 bp, Artificial Sequence
  DNA ligand.
acgcagtaagttgtaactaatgcgggggcgggg

SEQ ID NO: 36 - DNA, 33 bp, Artificial Sequence
  DNA ligand.
tgcgtcattcaacattgattacgcccccgcccc

SEQ ID NO: 37 - DNA, 34 bp, Artificial Sequence
  DNA ligand.
ccggcttttaagatcaacaacctggaaaggatca

SEQ ID NO: 38 - DNA, 34 bp, Artificial Sequence
  DNA ligand.
ggccgaaaattctagttgttggacctttcctagt

(19) United States
(12) Patent Application Publication
(10) Pub. No.: US 2011/0129935 A1
(43) Pub. Date: Jun. 2, 2011

(54) PROTEIN STABILITY ASSAY USING A FLUORESCENT REPORTER OF PROTEIN FOLDING

(75) Inventor: Patrick Schaeffer, Alice River (AU)

(73) Assignee: James Cook University, Townsville (AU)

(21) Appl. No.: 12/958,581

(22) Filed: Dec. 2, 2010

Related U.S. Application Data
(63) Continuation-in-part of application No. PCT/AU2009/001510, filed on Nov. 19, 2009.

(30) Foreign Application Priority Data
Nov. 19, 2008 (AU) 2008905981

(51) Int. Cl. G01N 33/50; C07H 21/00; C07K 14/00; G01N 21/64

(52) U.S. Cl. 436/86; 536/23.1; 530/350; 250/459.1

(57) ABSTRACT

The invention relates to methods and compositions for assessing protein stability, including improved assays for distinguishing between soluble and aggregated proteins. The methods and compositions include measuring residual fluorescence of a fusion protein in a soluble fraction as an indicator of protein solubility, and monitoring fluorescence quenching of a fusion protein as an indicator of protein stability. The fusion protein may comprise an amino acid sequence of a protein of interest, a peptide linker amino acid sequence and an amino acid sequence of a fluorescent marker protein.

### CROSS-REFERENCE TO RELATED APPLICATIONS

This application is a continuation-in-part of International Application No. PCT/AU2009/001510, filed Nov. 19, 2009, which claims priority to Australian Application No. 2008905981, filed Nov. 19, 2008, the contents of which are herein incorporated by reference in their entirety.

### FIELD OF THE INVENTION

THIS INVENTION relates to protein stability. More particularly, this invention relates to improved assays for distinguishing between soluble and aggregated proteins.

### REFERENCE TO SEQUENCE LISTING SUBMITTED ELECTRONICALLY

The official copy of the sequence listing is submitted electronically via EFS-Web as an ASCII formatted sequence listing with a file named 399284-sequences.txt, created on Dec. 2, 2010, and having a size of 7.28 KB and is filed concurrently with the specification. The sequence listing contained in this ASCII formatted document is part of the specification and is herein incorporated by reference in its entirety.

### BACKGROUND TO THE INVENTION

Proteins are of utmost importance to cellular function and must be, and remain, properly folded to perform their functions. Mutations and environmental factors can perturb the protein folding process, leading to the unfolding, change of conformation or misfolding of a protein. Usually defective proteins are cleared away by the proteasome. However, defective proteins can form multimeric protein aggregates, which can lead to protein folding/aggregation diseases.

Protein folding/aggregation diseases, which have proven particularly refractory to pharmaceutical development, are caused either by misfolding of a protein during biosynthesis subsequent to acquiring some mutation (Brown et al., J. Clin. Invest. 99:1432-44, 1997; Thomas et al., FEBS Lett. 312:7-9, 1992; Rao et al., Nature 367:639-42, 1994) or by aberrant protein processing leading to the formation of an aggregation-prone product, such as the peptide forming the amyloid plaques associated with Alzheimer's disease (Tan and Pepys, Histopathology 25:403-14, 1994; Harper and Lansbury, Annu. Rev. Biochem. 66:385-407, 1997), SOD1 in amyotrophic lateral sclerosis (Bruijn et al., Science 281:1851-53, 1998), α-synuclein in Parkinson's disease (Galvin et al., Proc. Natl. Acad. Sci. USA 96:13450-55, 1999), amyloid A and P deposits in systemic amyloidosis (Hind et al., J. Pathology 139:159-66, 1983), transthyretin fibrils in fatal familial insomnia (Colon and Kelly, Biochemistry 31:8654-60, 1992), and the intranuclear inclusions associated with polyglutamine expansions which cause Huntington's disease (Martin and Gusella, N. Engl. J. Med. 315:1267-76, 1986; Davies et al., Cell 90:537-48, 1997), spinocerebellar ataxia (Wells and Warren (eds.), “Genetic instabilities and hereditary neurological diseases,” Am J. Hum. Genet. 63(6):1921, 1998), spinobulbar muscular atrophy (La Spada et al., Nature 352:77-79, 1991), and Machado-Joseph Disease (Kawaguchi et al., Nature Genetics 8:221-28, 1994).

Proteins interact with one another, as well as with various ligands. These interactions are essential for the regulation of various cellular signaling pathways, and represent a large class of targets for drug discovery. Proteins have also become essential reagents in many industrial and scientific fields. For example, proteins are key players in proteomics and functional and structural genomics programs, with a goal to provide essential information for the design of improved pharmaceutical compounds.

However, existing assays for assessing protein stability are tedious, usually requiring lysis and fractionation of cells expressing the protein, followed by purification and protein analysis by SDS-polyacrylamide gel electrophoresis. Using traditional approaches, assessing protein stability upon exposure to a test condition, assessing changes in protein stability upon binding of a ligand, screening potential inhibitors of protein aggregation, and other procedures related to protein stability are inefficient and ill adapted to high-throughput screening.

### SUMMARY OF THE INVENTION

The present invention is directed to methods for assessing protein solubility/stability and/or a fusion protein suitable for use in such methods.

In one aspect, the invention provides a method for assessing protein solubility, the method comprising the steps of: a) exposing a fusion protein to a test condition, the fusion protein comprising a protein of interest (POI), a linker and a fluorescent marker protein, wherein the marker protein does not affect the solubility of the protein of interest; b) separating the fusion protein into soluble and insoluble fractions, wherein the soluble fraction comprises soluble fusion protein and the insoluble fraction comprises aggregates of the fusion protein; and c) measuring the residual fluorescence of the fusion protein in the soluble fraction as an indicator of protein solubility.

In another aspect, the invention provides a method for assessing protein stability, the method comprising the steps of: a) exposing a fusion protein to a test condition, the fusion protein comprising a protein of interest (POI), a linker and a fluorescent marker protein, wherein the marker protein does not affect the stability of the protein of interest; b) heating the fusion protein; and c) monitoring fluorescence quenching of the fusion protein as an indicator of protein stability.

In one embodiment, the methods further comprise the step of providing and/or purifying the fusion protein prior to exposing the fusion protein to a test condition.

Another embodiment includes producing a fusion protein by including the step of expressing a fusion protein in an expression system, wherein the expression system comprises a nucleic acid molecule encoding the fusion protein and a promoter active in the expression system operably linked to the nucleic acid molecule; and extracting a protein sample from the expression system, wherein the protein sample comprises the fusion protein.

The expression system can comprise an expression construct, wherein the nucleic acid molecule is operably linked to one or more regulatory sequences in the expression construct and the promoter is active in a host cell, and the fusion protein is expressed in the host cell.

The host cell can be a bacterial cell, an insect cell, a yeast cell, a nematode cell, or a mammalian cell.

Furthermore, the expression system can comprise an in vitro transcription/translation system.

In a further embodiment, producing a fusion protein comprises joining the protein of interest via the linker to the fluorescent marker protein.

Joining the protein of interest via the linker to the fluorescent marker protein can comprise ligating the protein of interest via the linker to the marker protein or the self-assembly of the protein of interest, the linker and the marker protein.

In a yet a further embodiment, the fluorescent marker protein is C-terminal to the protein of interest.

In an alternative embodiment, the fluorescent marker protein is N-terminal to the protein of interest.

These aspects include linkers of five to fifty or more amino acids connecting the protein of interest and the fluorescent marker protein.

Furthermore, these aspects extend to test conditions including, but not limited to, physical and chemical treatments, such as a change in temperature, a change in pH, a change in ionic strength, a change in salt concentration, addition of an oxidizing agent, addition of a reducing agent, addition of a detergent, as well as the addition of one or more ligands, for example, a protein, a metal ion or a small molecule, such as a pharmaceutical compound.

In some embodiments, exposing the fusion protein to a test condition occurs in a well of a microtiter plate. For example, a plurality of the same or different fusion proteins can be exposed to the same or different test conditions in separate wells of a microtiter plate.

In one embodiment of the aspect that includes a separation step, centrifugation is used to separate the fusion protein into soluble and insoluble fractions following exposure to a test condition.

In another embodiment of the aspect that includes a separation step, an aliquot of the fusion protein is spotted onto a selectively permeable matrix (e.g., a gel surface, such as an agarose gel surface or a polyacrylamide gel surface), following exposure to a test condition to separate the fusion protein into soluble and insoluble fractions.

The proteins of interest encompassed by these aspects include monomeric as well as multimeric (e.g., dimeric, trimeric and tetrameric) proteins.

These aspects extend to assessing protein solubility/stability upon exposure to a test condition, wherein the protein of interest is the protein whose solubility/stability is being assessed. Thus, in particular embodiments, the method for assessing protein solubility comprises a method for assessing protein stability.

In a further embodiment, the protein of interest is a mutant protein comprising one or more amino acid substitutions, insertions or deletions, and the method for assessing protein solubility comprises a method for assessing the stability of the mutant protein.

These aspects also extend to assessing changes in protein solubility/stability upon binding of a ligand, wherein the protein of interest is the protein whose solubility/stability is being assessed upon binding of the ligand. Thus, in certain embodiments, the method for assessing protein solubility comprises a method for screening for ligand binding by the protein of interest.

In a further embodiment, mutant versions of the protein of interest can be screened for ligand binding using this aspect of the invention.

Furthermore, these aspects of the invention extend to screening potential inhibitors of protein aggregation, wherein exposing the fusion protein to a test condition includes exposing the fusion protein to a potential inhibitor of protein aggregation, and wherein exposure to the potential inhibitor can occur before, after or simultaneously with exposure to the test condition. Thus, in certain embodiments, the method for assessing protein solubility comprises a method for screening potential inhibitors of protein aggregation.

In yet another aspect, the invention provides an isolated nucleic acid molecule comprising a polynucleotide encoding a peptide linker in-frame with a polynucleotide encoding a fluorescent protein, and an internal cloning site into which a heterologous polynucleotide encoding a protein of interest can be inserted in-frame with the linker and fluorescent protein coding sequences.

In one embodiment, the internal cloning site is upstream of the polynucleotide encoding a peptide linker in-frame with the polynucleotide encoding a fluorescent protein.

In an alternative embodiment, the internal cloning site is downstream of the polynucleotide encoding a fluorescent protein in-frame with the polynucleotide encoding a peptide linker.

In a further aspect, the invention provides a genetic construct comprising the isolated nucleic acid molecule of the above aspect.

The genetic construct can be an expression construct, wherein the isolated nucleic acid molecule is operably linked or connected to one or more regulatory sequences in an expression vector.

In yet a further aspect, the invention provides an isolated fusion protein comprising an amino acid sequence of a protein of interest, a peptide linker amino acid sequence and an amino acid sequence of a fluorescent marker protein.

In one embodiment, the peptide linker amino acid sequence comprises LGSGGH (SEQ ID NO:1).

In a further embodiment, the fluorescent marker protein is selected from the group consisting of green fluorescent protein, yellow fluorescent protein, blue fluorescent protein, red fluorescent protein, and orange fluorescent protein.

In still a further aspect, the invention provides a kit for assessing protein solubility and/or stability, as well as for screening potential inhibitors of protein aggregation, for use in the methods of the aforementioned aspects. In one embodiment, the kit includes an expression vector comprising a polynucleotide encoding a peptide linker in-frame with a polynucleotide encoding a fluorescent protein, and an internal cloning site into which a heterologous polynucleotide encoding a protein of interest can be inserted in-frame with the linker and fluorescent protein coding sequences.

In one embodiment, the kit comprises one or more oligonucleotide primer pairs for introducing a promoter, a ribosomal binding site, and a linker for generating a fusion gene comprising a gene coding for the protein of interest joined in-frame with the fluorescent marker gene, the kit further comprising one or more reagents necessary to carry out in vitro amplification reactions.

### BRIEF DESCRIPTION OF THE FIGURES

FIG. 1. Fluorescent marker proteins as a probe for protein unfolding and aggregation.

(a) Reaction coordinates of irreversible protein aggregation. ΔG* is the change in free energy of activation.

(b) Principle of GFP-based stability assay (GFP-Basta). The thermal denaturation of protein of interest-GFP fusion proteins produces a heterogeneous population of folded and denatured fluorescent proteins. The fraction of denatured proteins form aggregates, which are further discarded from the solution to allow the measurement of the soluble fraction F_{fold}.

FIG. 2. Comparison of thermal aggregation profiles of Tus, Tus-GFP, chloramphenicol acetyl transferase (CAT), CAT-GFP, glycerol kinase (GK), GK-GFP, and GFP.

(a) Equal amounts of Tus, Tus-GFP and GFP were mixed, heat treated during 5 minutes at temperatures ranging from 25° C. to 53.3° C., centrifuged to discard the aggregates, and electrophoretically separated by SDS-PAGE. Fluorescence (top gel) was recorded with illumination at 365 nm, before Coomassie blue staining (bottom gel).

(b) Protein bands were quantified using ImageJ and thermal aggregation profiles were plotted, fitted and used to determine the T_{agg} of proteins. Tus-GFP and GFP thermal aggregation profiles were obtained both by SDS-PAGE and the S method. The T_{agg} of Tus and Tus-GFP were 45.4±0.2 and 44.2±0.4° C. (44.3±0.2° C., S method), respectively. The T_{agg} value of GFP was 79.6±0.6° C.

(c) CAT, CAT-GFP and GFP were separately heat treated during 5 minutes at temperatures ranging from 25° C. to 78.1° C., centrifuged as before and loaded on separate gels (top gel). GK, GK-GFP and GFP were separately heat treated during 30 minutes at temperatures ranging from 42° C. to 64° C., centrifuged as before and loaded on separate gels (bottom gel).

(d) Thermal aggregation profiles of CAT-GFP, CAT, GK, and GK-GFP were obtained by SDS-PAGE. The T_{agg} of CAT-GFP, CAT, GK-GFP, and GK were 68.8±0.4, 67.9±0.2, 51.1±0.3 and 50.4±0.3° C., respectively.

FIG. 3. Isothermal aggregation reactions.

Effect of various additives on Tus-GFP and GFP (inset) stability. k_{agg} values were determined at 46° C. by the S method. Additives were present at final concentrations of either 25% glycerol, 0.3 M (NH₄)₂SO₄, 0.3 M NaCl, 0.4 M KCl, or 0.3 M MgCl₂ in 0.5× Buffer B.

FIG. 4. Thermal aggregation profiles of Tus-GFP and Tus-GFP-Ter complex.

(a) EMSA of thermally denatured Tus-GFP-Ter complex. The bottom bands correspond to TerB bound Tus-GFP.

(b) Thermal aggregation profiles of Tus-GFP incubated with or without TerB were obtained by EMSA (T_{agg}=58.7±0.6° C.) or S method (T_{agg}=58.7±0.7° C.). The double-headed arrow indicates a ligand-induced ΔT_{agg} of 14.4° C.

FIG. 5. Correlation between k_{agg} and K_{D}.

(a) k_{agg} of Tus-GFP in complex with Ter variants (TerB, Ter-AG, Ter-AAG, or TT-lock) were determined at 50° C. by the S method.

(b) Correlation between ln(K_{D}) from published SPR data (Mulcair et al., Cell 125:1309-19, 2006) and the ln(k_{agg(Tus)}/k_{agg(Tus-Ter)}).

FIG. 6. Rapid throughput screening using the S method.

(a) F_{fold} of Tus-GFP in the presence of additives (same buffer conditions as in FIG. 3) or TerB (+Ter).

(b) F_{fold} of CAT-GFP in the presence of 10 mM chloramphenicol (Chlor) or 9.2 mM ampicillin (Amp).

(c) F_{fold} of GK-GFP in the presence of 1 mM glycerol (+gly) or 1 mM glucose (+glu).

(d) Effect of CAT or BSA on the F_{fold} of GK-GFP in the presence of 1 mM glycerol (+gly) or 1 mM glucose (+glu).

Each set of reactions was performed with a control (Ctrl) prepared in exactly the same buffer conditions minus additive or ligand. All reactions were done in triplicate. Error bars indicate SEM.

FIG. 7. pH-dependence of Tus-GFP stability.

(a) Tus-GFP and (b) GFP were incubated at 46° C. for 2 minutes, 5 minutes and 10 minutes in phosphate buffers of 5 different pHs (between 6.7 and 8) to determine k_{agg} as a function of pH. The bottom panel summarizes the k_{agg}, their 95% confidence interval and the R-square value (n=3).

FIG. 8. Comparison of melting curves obtained with 2.5 μM Tus-GFP fusion protein (bottom traces) versus a Tus (2.5 μM) and GFP (2.5 μM) mixture (top traces).

The first dip in the bottom traces corresponds to the unfolding and aggregation of the Tus in the fusion protein, while the second dip corresponds to the loss of fluorescence of GFP at temperatures higher than 80° C.

FIG. 9. Comparison of transformed melting curves obtained with 2.5 μM Tus-GFP fusion protein versus a Tus (2.5 μM) and GFP (2.5 μM) mixture.

Transformed traces illustrate that a peak at a T_{agg} of 50° C. can only be obtained with the Tus-GFP fusion protein.

FIG. 10. Comparison of melting curves obtained with 2.5 μM Tus-GFP in the presence of an excess concentration of TerC and increasing concentrations of KCl (150 mM-350 mM) to analyse the effect of ionic strength on the Tus-TerC interaction.

The first dip in each of the traces corresponds to the unfolding and aggregation of the Tus in the fusion protein, while the second dip corresponds to the loss of fluorescence of GFP at temperatures higher than 75° C.

### BRIEF DESCRIPTION OF THE SEQUENCE LISTING

SEQ ID NO:1 Amino acid sequence of a linker peptide.
SEQ ID NO:2 Nucleotide sequence of a cloning linker.
SEQ ID NO:3 Nucleotide sequence of a synthetic DNA cloning sequence.
SEQ ID NO:4 Nucleotide sequence of a synthetic DNA cloning sequence.
SEQ ID NOs:5-8 Nucleotide sequences of PCR primers.
SEQ ID NOs:9-38 Nucleotide sequences of DNA ligands.

### DETAILED DESCRIPTION OF THE INVENTION

The present invention relates to improved methods for ascertaining a protein's solubility/stability, including changes thereto, under a variety of conditions, including, but not limited to, physical and chemical treatments, such as a change in temperature, a change in pH, a change in ionic strength, a change in salt concentration, addition of an oxidizing agent, addition of a reducing agent, addition of a detergent, as well as the addition of one or more ligands, for example, a protein, a metal ion or a small molecule, such as a pharmaceutical compound.

Throughout this specification, unless the context requires otherwise, the words “comprise”, “comprises” and “comprising” will be understood to imply the inclusion of a stated integer or group of integers but not the exclusion of any other integer or group of integers.

In one aspect, the invention provides a method for assessing protein solubility, the method comprising the steps of: a) exposing a fusion protein to a test condition, the fusion protein comprising a protein of interest, a linker and a fluorescent marker protein, wherein the marker protein does not affect the solubility of the protein of interest; and b) separating the fusion protein into soluble and insoluble fractions, wherein the soluble fraction comprises soluble fusion protein and the insoluble fraction comprises aggregates of the fusion protein; and d) measuring the residual fluorescence of the fusion protein in the soluble fraction as an indicator of protein solubility.

In another aspect, the invention provides a method for assessing protein stability, the method comprising the steps of: a) exposing a fusion protein to a test condition, the fusion protein comprising a protein of interest (POI), a linker and a fluorescent marker protein, wherein the marker protein does not affect the stability of the protein of interest; b) heating the fusion protein; and c) monitoring fluorescence quenching of the fusion protein as an indicator of protein stability.

Preferably, the methods further comprise the step of providing and/or purifying the fusion protein prior to exposing the fusion protein to a test condition.

An aspect of the present invention is the discovery that in a fusion protein comprising a protein of interest and a fluorescent marker protein separated by an appropriate linker, the unfolding properties of the two proteins are uncoupled and occur as independent events depending on their individual stabilities. That is, the fluorescent marker protein does not affect the stability of the protein of interest.

In one embodiment, producing a fusion protein comprises expressing a fusion protein in an expression system, wherein the expression system comprises a nucleic acid molecule encoding the fusion protein and a promoter active in the expression system operably linked to the nucleic acid molecule; and extracting a protein sample from the expression system, wherein the protein sample comprises the fusion protein.

By “protein” is meant an amino acid polymer. The amino acids can be natural or non-natural amino acids, D- or L-amino acids as are well understood in the art.

The term “protein” includes and encompasses “peptide”, which is typically used to describe a protein having no more than fifty (50) amino acids and “polypeptide”, which is typically used to describe a protein having more than fifty (50) amino acids.

As used herein, “fusion protein” describes a protein formed by the joining of two or more individual proteins to produce into a contiguous or fused protein in which the two or more individual proteins retain their individual activities. This term includes a protein formed by way of ligation or self-assembly of two or more individual proteins, as well as an expressed protein resulting from the joining of two or more genes or gene fragments.

Fusion proteins can be produced using any number of ligation and/or self-assembly methodologies, as are well known to one of skill in the art. Exemplary protein ligation techniques include reductive amination, diazo coupling, thioether bond, disulfide bond, amidation, thiocarbamoyl chemistries, sortase-mediated ligation (Mao et al., J. Am. Chem. Soc. 126:2670-71, 2004), expressed protein ligation utilizing intein domains (Pickin et al., J. Am. Chem. Soc. 130:5667-69, 2008; Seyedsayamdost et al., Nat Protoc. 5:1225-35, 2007), and ligation reactions between thioester peptides and bis-cysteinyl linkers (Ziaco et al., Org. Lett., 10:1955-58, 2008).

Self-assembly techniques for the production of fusion proteins are likewise well known and include, for example, the assembly of proteins individually labelled with avidin/streptavidin and biotin.

Fusion proteins can also be produced by linking at least a first nucleic acid molecule encoding at least a first amino acid sequence to at least a second nucleic acid molecule encoding at least a second amino acid sequence, so that the encoded sequences are translated as a contiguous amino acid sequence either in vitro or in vivo. Fusion protein design and expression are well known in the art, and methods of fusion protein expression are described, for example, in U.S. Pat. No. 5,935,824.

By “linker” is meant a segment that functionally joins two amino acid sequences in a fusion protein. The term “functionally joins” denotes a connection between the two amino acid sequences in the fusion protein that maintains and/or facilitates proper folding (and hence function) of each of the sequences. Linkers can include amino acids, including amino acids capable of forming disulfide bonds, but can also include other molecules such as, for example, polysaccharides or fragments thereof. For example, as described herein, a linker joins a protein of interest to a fluorescent marker protein in a fusion protein. The linker can be C-terminal to the protein of interest and N-terminal to the fluorescent marker protein in the fusion protein. Alternatively, the linker can be C-terminal to the fluorescent marker protein and N-terminal to the protein of interest in the fusion protein.

The linker used in the fusion protein is such that the unfolding and aggregation state of the protein of interest is not tied to the activity of the fluorescent marker protein. For example, the linker is such that destabilisation of the protein of interest (including unfolding, aggregation and/or changes in solubility) in the fusion protein in response to a test condition does not affect the folding activity required for fluorescence of the fluorescent marker protein.

Such a linker can be any length, so long as the unfolding properties of the two proteins in the fusion protein (i.e., the protein of interest and the fluorescent marker protein) are uncoupled and occur as independent events. For example, the linker can comprise 4, 5, 6, 7, 8, 9, 10, 12, 15, 20, 25, 30, 35, 40, 45, 50, or more amino acids. In one embodiment, the linker is a six amino acid linker: LGSGGH (SEQ ID NO:1).

In certain embodiments, the fluorescent marker protein is joined C-terminal to the protein of interest via the linker in the fusion protein. In other embodiments, the fluorescent marker protein is joined N-terminal to the protein of interest via the linker in the fusion protein.

By “protein of interest” is meant any protein, including monomeric as well as multimeric (e.g., dimeric, trimeric, tetrameric, and pentameric) proteins, for which an assessment of solubility in response to one or more test conditions is desired. The term also encompasses a mutant protein comprising one or more amino acid substitutions, insertions and/or deletions compared to its wild-type counterpart.

Proteins of interest encompassed by the invention include monomeric as well as multimeric (e.g., dimeric, trimeric, tetrameric, and pentameric) proteins. Exemplary proteins of interest include, but are not limited to: amyloid proteins, such as amyloid A, amyloid P and β-amyloid peptide, superoxide dismutase 1, presenillin 1 and 2, α-synuclein, cystic fibrosis transmembrane conductance regulator, transthyretin, amylin, lysozyme, gelsolin, p53, rhodopsin, insulin, insulin receptor, fibrillin, α-ketoacid dehydrogenase, collagen, keratin, prion protein, immunoglobulins, atrial natriuretic peptide, seminal vesicle exocrine protein, β2-microglobulin, precalcitonin, ataxin 1, ataxin 2, ataxin 3, ataxin 6, ataxin 7, Huntingtin, androgen receptor, CREB-binding protein, dentaorubral pallidoluysian atrophy-associated protein, maltose-binding protein, ABC transporter, glutathione S transferase, and thioredoxin.

Mutant proteins can be produced by a variety of standard, mutagenic procedures known to one of skill in the art. A mutation can involve the modification of the nucleotide sequence of a single gene, blocks of genes or a whole chromosome, with the subsequent production of one or more mutant proteins. Changes in single genes may be the consequence of point mutations which involve the removal, addition or substitution of a single nucleotide base within a DNA sequence, or they may be the consequence of changes involving the insertion or deletion of large numbers of nucleotides.

Mutations occur following exposure to chemical or physical mutagens. Such mutation-inducing agents include ionizing radiation, ultraviolet light and a diverse array of chemical agents, such as alkylating agents and polycyclic aromatic hydrocarbons, all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation, which can subsequently be reflected at the protein level. Mutation also can be site-directed through the use of particular targeting methods.

Mutagenic procedures of use in producing mutant proteins for study according to the methods disclosed herein include, but are not limited to, random mutagenesis (e.g., insertional mutagenesis based on the inactivation of a gene via insertion of a known DNA fragment, chemical mutagenesis, radiation mutagenesis, error prone PCR (Cadwell and Joyce, PCR Methods Appl. 2:28-33, 1992)) and site-directed mutagenesis (e.g., using specific oligonucleotide primer sequences that encode the DNA sequence of the desired mutation). Additional methods of site-directed mutagenesis are disclosed in U.S. Pat. Nos. 5,220,007; 5,284,760; 5,354,670; 5,366,878; 5,389,514; 5,635,377; and 5,789,166.

The term “fluorescent marker protein” includes a protein that, in response to incident radiation in the visible or ultraviolet spectra, emits radiation at a wavelength longer than the incident radiation. The term “fluorescent domain” is used to indicate the portion of a fluorescent protein having a structure distinct from an adjacent portion(s) of the protein and which is responsible for the fluorescence. In practice, fluorescent proteins and fluorescent protein domains generally emit in the visible portion of the spectrum.

Fluorescent marker proteins are well know in the art. These include fluorescent proteins derived from the jellyfish Aequorea victoria, for example, green fluorescent protein (GFP) and its variants, such as yellow fluorescent protein (YFP) and blue (or cyan) fluorescent protein (BFP) (see, e.g., Waldo et al., Nat. Biotechnol. 17:691-95, 1999; Tsien, R. Y. Annu. Rev. Biochem. 67:509-44, 1998; Griesbeck et al., J. Biol. Chem. 276:29188-94, 2001; Zacharias et al., Science 296:913-16, 2002; Nagai et al., Nat. Biotechnol. 20:87-90, 2002; Nguyen et al., Nat. Biotechnol. 23:355-60, 2005; Rizzo et al., Nat. Biotechnol. 22:445-49, 2004). Also included are fluorescent proteins derived from Discosoma sp., for example, red fluorescent protein (RFP) and orange fluorescent protein (OFP) (Wang et al., Proc. Natl. Acad. Sci. USA 101:16745-49, 2004; Sharer et al., Nat. Biotechnol. 22:1567-72, 2004; U.S. Pat. No. 7,193,052), as well as an OFP derived from Fungia concinna (Karasawa et al., Biochem. J. 381:307-12, 2004). Additionally, fluorescent marker proteins include proteins that require a co-factor to fluoresce, such as luciferase.

Following production of a fusion protein, a purification step can be performed to separate the fusion protein from the two or more individual proteins that were joined to produce the fusion protein. One method for purification, involving ultrafiltration in the presence of ammonium sulfate, is described in U.S. Pat. No. 6,146,902. Alternatively, fusion proteins can be purified away from unreacted individual proteins by any number of standard techniques including, for example, size exclusion chromatography, density gradient centrifugation, hydrophobic interaction chromatography, or ammonium sulfate fractionation. See, for example, Anderson et al., J. Immunol. 137:1181-86, 1986 and Jennings & Lugowski, J. Immunol. 127:1011-18, 1981. The composition and purity of the fusion protein can be determined by GLC-MS and MALDI-TOF spectrometry.

As described herein, a fusion protein of the invention can be expressed in an expression system, wherein the expression system comprises a nucleic acid molecule encoding the fusion protein and a promoter active in the expression system operably linked to the nucleic acid molecule.

The term “expression system” as used herein designates a system that comprises a nucleic acid molecule encoding a fusion protein of the invention, a promoter active in the expression system operably linked to the nucleic acid molecule and the necessary biological and/or chemical elements to allow for transcription and translation of the nucleic acid molecule.

By “nucleic acid molecule” is meant single- or double-stranded mRNA, RNA, cRNA, and DNA inclusive of cDNA and genomic DNA.

In one embodiment, the expression system comprises an expression construct,

wherein the nucleic acid molecule is operably linked to one or more regulatory sequences in the expression construct and the promoter is active in a host cell, and the fusion protein is expressed in the host cell.

By “expression construct” is meant a genetic construct wherein the nucleic acid molecule to be expressed is operably linked or operably connected to one or more regulatory sequences in an expression vector.

An “expression vector” can be either a self-replicating extra-chromosomal vector such as a plasmid, or a vector that integrates into a host genome.

In one aspect of the invention, the expression vector is a plasmid vector.

By “operably linked” or “operably connected” is meant that the regulatory sequence(s) is/are positioned relative to the nucleic acid molecule to be expressed to initiate, regulate or otherwise control expression of the nucleic acid molecule.

Regulatory sequences will generally be appropriate for the host cell used for expression. Numerous types of appropriate expression vectors and suitable regulatory sequences are known in the art for a variety of host cells.

One or more regulatory sequences can include, but are not limited to, promoter sequences, leader or signal sequences, ribosomal binding sites, transcriptional start and termination sequences, translational start and termination sequences, splice donor/acceptor sequences, and enhancer or activator sequences.

Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes, such as 3-phosphoglycerate kinase (PGK), and the acid phosphatase promoter. Exemplary eukaryotic promoters include the CMV immediate early promoter, the HSV thymidine kinase promoter, heat shock promoters, the early and late SV40 promoter, LTRs from retroviruses, and the mouse metallothionein-I promoter.

Constitutive or inducible promoters as known in the art can be used and include, for example, tetracycline-repressible, IPTG-inducible, alcohol-inducible, acid-inducible and/or metal-inducible promoters.

In one aspect, the expression vector comprises a selectable marker gene. Selectable markers are useful whether for the purposes of selection of transformed bacteria (such as bla, kanR and tetR) or transformed mammalian cells (such as hygromycin, G418 and puromycin).

Suitable host cells for expression can be prokaryotic or eukaryotic, such as E. coli (DH5α for example), yeast cells, SF9 cells utilized with a baculovirus expression system, nematode cells, or any of various mammalian or other animal host cells, without limitation thereto.

Introduction of expression constructs into suitable host cells can be by way of techniques including, but not limited to, electroporation, heat shock, calcium phosphate precipitation, DEAE dextran-mediated transfection, liposome-based transfection (e.g., lipofectin, lipofectamine), protoplast fusion, microinjection or microparticle bombardment, as are well known in the art.

Cells can be harvested by centrifugation, disrupted by physical or chemical means, and the resulting crude extract comprising the fusion protein is exposed to one or more test conditions, or retained for further purification. Microbial cells employed for expression of proteins can be disrupted by any convenient method, including freeze-thaw cycling, sonication, mechanical disruption, or use of cell lysing agents. Such methods are well known to those skilled in the art.

The expressed fusion proteins can be recovered and purified from recombinant cell cultures by methods including ammonium sulfate or ethanol precipitation, acid extraction, nickel affinity chromatography (Ni-NTA), anion or cation exchange chromatography, phosphocellulose chromatography, hydrophobic interaction chromatography, affinity chromatography, hydroxylapatite chromatography, lectin chromatography, and gel filtration. Protein refolding steps can be used, as necessary, in completing configuration of the polypeptide. If desired, high performance liquid chromatography (HPLC) can be employed for final purification steps.

In another embodiment, the expression system comprises the in vitro production of a fusion gene comprising a gene coding for the protein of interest fused in-frame with the linker and fluorescent marker gene. Methods of in vitro production of a fusion gene are well known in the art, and include, for example, overlap extension PCR, which utilizes one or more oligonucleotide primer pairs to introduce a promoter, a ribosomal binding site and a linker sequence for generating the fusion gene. Expression of the fusion protein from the fusion gene can be accomplished using a cell-free transcription/translation system. Cell-free translation systems can use mRNAs transcribed from a DNA construct comprising a promoter operably linked to a nucleic acid encoding a fusion protein of the invention. In some aspects, the DNA construct can be linearized prior to conducting an in vitro transcription reaction. The transcribed mRNA is then incubated with an appropriate cell-free translation extract, such as an E. coli extract, a rabbit reticulocyte extract, or a wheat germ reticulocyte extract to produce the desired fusion protein.

It is to be understood that similar protein expression and purification methods can be used to prepare samples of proteins of interest that lack the fluorescent marker protein. Purified samples of a protein of interest with and without the fluorescent marker protein can be used in assays to determine if the fluorescent marker protein has a significant effect on the behaviour (e.g., the unfolding properties) of the protein of interest in a fusion protein in response to various test conditions. For example, melting curves of purified samples of the protein of interest with and without the fluorescent marker protein can be generated under various denaturing conditions. In those instances where a significant effect is observed, a correction factor can be determined (by reference to the melting curves) to compensate for the effect.

Fusion proteins of the invention can be exposed or subjected to a test condition. The term “test condition” refers to a substance, compound, molecule, mixture, or treatment with which the fusion protein can be contacted or treated, for purposes of evaluating the effect thereof on the fusion protein. The effect thereof on the fusion protein to be evaluated can include the unfolding or denaturing of that portion of the fusion protein that corresponds to the protein of interest.

Test conditions include, but not limited to, physical and chemical treatments, such as temperature (e.g., 25° C. to 100° C., such as 30° C., 35° C., 40° C., 45° C., 50° C., 55° C., 60° C., 65° C., 70° C., 75° C., 80° C., 85° C., 90° C., and 95° C.), pH (e.g., between 0 and 14, inclusive, such as 1, 2, 3, 4, 5, 6, 7, 8, 9, 10, 11, 12, and 13), ionic strength, salt (e.g., NaCl, KCl, CaCl₂, (NH₄)₂SO₄, MgCl₂, and MgSO₄) concentration (e.g., between 50 mM and 500 mM, such as 100 mM, 150 mM, 200 mM, 250 mM, 300 mM, 350 mM, 400 mM, and 450 mM), an oxidizing agent (e.g., nitric acid, peroxides, sulfoxides, permanganate salts, hypochlorite, chlorite, chlorate, perchlorate, and halogens), a reducing agent (e.g., hydrogen, metals, and hydrocarbons), a detergent (e.g., N-Lauroylsarcosine, sodium dodecyl sulfate, sodium deoxycholate, TWEEN® 20, TWEEN® 80, Triton® X-100, saponin, CHAPS, Nonidet™ P 40, Poly(ethylene glycol), Polysorbate 20, Polysorbate 60, and Polysorbate 80), as well as the addition of one or more ligands, for example, a protein, a metal ion or a small molecule, such as a pharmaceutical compound.

To evaluate the effect of multiple test conditions on one or more fusion proteins, any number of high-throughput assays can be utilized. The assays are designed to screen large combinations of fusion proteins and test conditions by automating the assay steps and providing the necessary components from any convenient source to assay, which are typically run in parallel (e.g., in microtiter formats using robotic assays). Thus, by using high-throughput assays it is possible to screen several thousand different test condition/fusion protein combinations in a short period of time, for example, 24 hours. In particular, each well of a microtiter plate can be used to run a separate assay against a selected test condition to evaluate the effect thereof on the same fusion protein, or, alternatively, each well of the microtiter plate can be used to run a separate assay against a selected fusion protein to evaluate the effect thereof on of the same test condition. As will be understood by one of skill in the art, various groupings (including multiple wells of the same fusion protein/test condition to provide duplicates) and arrangements on the microtiter plate are useful in high-throughput assays.

For example, robotic high-throughput systems for screening multiple test conditions on one or more fusion proteins typically include a robotic armature which transfers fluid from a source to a destination, a controller which controls the robotic armature, a detector, a data storage unit, and an assay component such as a microtiter dish comprising a well that includes a fusion protein. A number of robotic fluid transfer systems are available, or can easily be made from existing components. For example, commercially-available robotics systems (e.g., TekCel Corporation, Hopkinton, Md., USA) can be used to set up several parallel simultaneous high-throughput systems.

Thus, in some embodiments, exposing the fusion protein to a test condition occurs in a well of a microtiter plate. For example, a plurality of the same or different fusion proteins can be exposed to the same or different test conditions in separate wells of a microtiter plate.

In some embodiments, following exposure of a fusion protein of the invention to a test condition, centrifugation is used to separate the fusion protein into soluble and insoluble fractions, wherein the soluble fraction comprises soluble fusion protein and the insoluble fraction comprises aggregates of the fusion protein.

Diffusion through a selectively permeable matrix can be used to separate the fusion protein into soluble and insoluble fractions following exposure to a test condition, wherein the soluble fraction comprises soluble fusion protein and the insoluble fraction comprises aggregates of the fusion protein. Specifically, one or more aliquots of the fusion protein can be spotted onto the surface of a selectively permeable matrix following exposure to a test condition to separate the fusion protein into soluble and insoluble fractions.

Well known selectively permeable matrices can comprise aqueous gels or various types of sediment or fibrous substances. The most conventional matrix is one in which a gel is used, in particular an agar or an agarose gel, suitably comprising a buffered 1%-solution of agar or agarose which is permitted to solidify prior to the application of a fusion protein. Additional polymers useful in forming selectively permeable matrices include, for example, polyacrylamide, poly(α-hydroxy acids) such as polylactic acid (PLA), polyglycolic acid (PGA) and copolymers thereof (PLGA), poly(orthoesters), polyurethanes, and hydrogels, such as polyhydroxyethyl methacrylate (poly-HEMA) or polyethylene oxide-polypropylene oxide copolymer (PEO-PPO).

By “selectively permeable” is meant that soluble proteins are able to enter the matrix, while protein aggregates are unable to. For example, in 1% agarose gel, aggregates larger than about 0.4 μm are unable to enter the gel. Methods of controlling the permeability of a matrix (e.g., by varying the amount of matrix material and/or including various cross-linking reagents) are well know in the art.

In other embodiments, following exposure of a fusion protein of the invention to a test condition the fusion protein is heated, the fluorescent marker protein acting as a probe to monitor unfolding and/or aggregation of the protein of interest through fluorescent marker protein fluorescence quenching occurring upon unfolding and/or aggregation of the protein of interest.

As will be understood by one of skill in the art, the step of heating the fusion protein and monitoring/measuring fluorescence quenching following exposure to a test condition can be performed in a manner that promotes high-throughput analysis of multiple test conditions on one or more fusion proteins. For example, a thermocycler can be used to heat the fusion protein and monitor/measure fluorescence quenching.

In certain embodiments, the method for assessing protein solubility described herein encompasses a method for assessing protein stability upon exposure to one or more test conditions, wherein the protein of interest is the protein whose stability is being assessed.

In other embodiments, the method for assessing protein solubility described herein encompasses a method for screening potential inhibitors of protein aggregation, wherein exposing the fusion protein to a test condition includes exposing the fusion protein to a potential inhibitor of protein aggregation, and wherein exposure to the potential inhibitor can occur before, after or simultaneously with exposure to the test condition.

By “potential inhibitors of protein aggregation” is intended a molecule to be tested for its ability to inhibit protein aggregation using the methods described herein. Examples of such molecules include, but are not limited to, peptides, nucleic acids, carbohydrates, and small molecules. The term is meant to encompass both natural compounds (e.g., purified from a biological source) as well as synthetic compounds.

In particular embodiments, the method for assessing protein solubility described herein encompasses a method for assessing changes in protein stability upon binding of a ligand, wherein the protein of interest is the protein whose stability is being assessed upon binding of the ligand.

As used herein, the term “ligand” refers to an agent that binds a protein of interest, and includes without limitation metals, peptides, proteins (e.g., protein-protein interactions), lipids, polysaccharides, nucleic acids, and small organic molecules. Complex mixtures of substances such as natural product extracts, which can include more than one ligand, are also encompassed, and the component(s) that binds the protein of interest can be purified from the mixture in a subsequent step.

The ligand can bind the protein of interest when the protein of interest is in its native conformation, when it is partially or totally unfolded or denatured, or when it is partially or totally aggregated.

In a further embodiment, mutant versions of the protein of interest can be screened for ligand binding using this aspect of the invention. For example, libraries of mutant proteins can be screened for variants with increased (or decreased) affinity for a ligand, as compared to a wild-type protein of interest. Alternatively, libraries of mutant proteins can be screened for variants with improved stability or function generally (or in the presence of one or more test conditions), relative to a wild-type protein of interest.

In yet another aspect, the invention provides an isolated nucleic acid molecule comprising a polynucleotide encoding a peptide linker in-frame with a polynucleotide encoding a fluorescent protein, and an internal cloning site into which a heterologous polynucleotide encoding a protein of interest can be inserted in-frame with the linker and fluorescent protein coding sequences.

In one embodiment, the internal cloning site is upstream of the polynucleotide encoding a peptide linker in-frame with the polynucleotide encoding a fluorescent protein. In an alternative embodiment, the internal cloning site is downstream of the polynucleotide encoding a fluorescent protein in-frame with the polynucleotide encoding a peptide linker.

In a further aspect, the invention provides a genetic construct comprising the isolated nucleic acid molecule of the above aspect.

The genetic construct can be an expression construct, wherein the isolated nucleic acid molecule is operably linked or connected to one or more regulatory sequences in an expression vector as described herein.

In yet a further aspect, the invention provides an isolated fusion protein comprising an amino acid sequence of a protein of interest, a peptide linker amino acid sequence and an amino acid sequence of a fluorescent marker protein, as described herein.

By “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

In still a further aspect, the invention provides a kit for assessing protein solubility and/or stability, as well as for screening potential inhibitors of protein aggregation, for use in the methods of the aforementioned aspects. In one embodiment, the kit includes an expression vector comprising a polynucleotide encoding a peptide linker in-frame with a polynucleotide encoding a fluorescent protein, and an internal cloning site into which a heterologous polynucleotide encoding a protein of interest can be inserted in-frame with the linker and fluorescent protein coding sequences. The expression vector can include one or more regulatory sequences as described herein.

In a further embodiment, the kit comprises one or more oligonucleotide primer pairs for introducing a promoter, a ribosomal binding site, and a linker sequence for generating a fusion gene comprising a gene coding for the protein of interest fused in-frame with the fluorescent marker gene; the kit further comprising one or more reagents necessary to carry out in vitro amplification reactions, including DNA sample preparation reagents, appropriate buffers (for example, polymerase buffer), salts (for example, magnesium chloride), and deoxyribonucleotides (dNTPs).

In such a kit, an appropriate amount of the aforementioned one or more oligonucleotide primer pairs is provided in one or more containers, or held on a substrate. An oligonucleotide primer can be provided in an aqueous solution or as a freeze-dried or lyophilized powder, for instance. The container(s) in which the oligonucleotide(s) are supplied can be any conventional container that is capable of holding the supplied form, for instance, microfuge tubes, ampoules, or bottles. In some applications, pairs of primers are provided in pre-measured single use amounts in individual (typically disposable) tubes or equivalent containers. With such an arrangement, the polynucleotide encoding a protein of interest can be added to the individual tubes and overlap extension PCR carried out directly, followed by in vitro transcription/translation of the fusion gene comprising a gene coding for the protein of interest fused in-frame with the linker/fluorescent marker gene.

The amount of each oligonucleotide primer pair supplied in the kit can be any appropriate amount, and can depend on the market to which the product is directed. General guidelines for determining appropriate amounts can be found, for example, in Sambrook et al., Molecular Cloning: A Laboratory Manual, Cold Spring Harbor Laboratory Press, Cold Spring Harbor, N.Y., 2001; Ausubel et al. (eds.), Short Protocols in Molecular Biology, John Wiley and Sons, New York, N.Y., 1999; and Innis et al., PCR Applications, Protocols for Functional Genomics, Academic Press, Inc., San Diego, Calif., 1999.

So that the invention may be readily understood and put into practical effect, the following non-limiting Examples are provided.

### EXAMPLES

### Experimental Procedures

### Cloning

The plasmid pPMS1259 coding for the Tus-GFP fusion protein contains a sequence coding for a His₆-Tag followed by the E. coli Tus coding sequence, a sequence coding for a linker and a GFPuv coding sequence. This vector is based on the pET vector backbone containing the T7 RNA polymerase promoter and a ribosome-binding site. The linker coding sequence separating Tus and GFP consists of: 5′-AATTTGGGATCCGGCGGTCATATGACT-3′ (SEQ ID NO:2).

For construction of the plasmid pMM001 encoding the Tus protein and the linker sequence, a stop codon (TGA) was introduced directly downstream of the linker in pPMS1259 by the following DNA manipulation. The plasmid pPMS1259 was digested with NdeI resulting in the linearization of the plasmid and the loss of a 271-bp fragment of the GFP gene. The 5′-overhangs were end-filled with Phusion DNA polymerase (Finnzymes, Espoo, Finland) resulting in the deletion of the NdeI sites. Owing to the presence of an adenosine nucleotide following both NdeI sites, a TGA stop codon was created upon recircularization of the end-filled plasmid by T4 DNA ligase to generate pMM001.

The plasmid pMM001 was next transformed into DH5α, plated on plates supplemented with 100 μg/ml ampicillin for overnight growth. Successful ligation was verified by NdeI-BamHI double digestion of amplified plasmid from transformants using a mini-prep kit (Axy Prep Plasmid, Axygen, Union City, Calif., USA), PCR reaction on pPMS1259 and the new construct pMM001 using primers specific for upstream and downstream sequences of the Tus-GFP gene, and comparison of resulting fragment sizes, and by a NdeI-BamHI double digestion of the PCR products obtained from transformants. Expected bands were obtained for each control, and the plasmid was used for the expression of the Tus protein with the linker.

A GFP cloning cassette was designed to produce CAT and GK fusion proteins. For this, the plasmid pET-20b(+) from Novagen (San Diego, Calif., USA) was digested by NdeI and EcoRI and ligated with a double stranded synthetic DNA (sense: TATGCACCACCACCACCACCACGATATCGCCAAACTTAAGGCCGG CGCTAGCTTGGGATCCGGCGGTCATATgACTAGTGCCAAAAAG (SEQ ID NO:3); anti-sense: AATTCTTTTTGGCACTAGTCATATGACCGCCGGATCCC AAGCTAGCGCCGGCCTTAAGTTTGGCGATATCGTGGTGGTGGTGGTGGTGCA (SEQ ID NO:4)) comprising NdeI and EcoRI overhangs and 4 internal restriction sites (EcoRV, AflII, NheI, and SpeI; underlined in the sense primer) yielding pET-A. A GFP coding sequence was obtained by SpeI/EcoRI digest of pPMS1259 and was ligated with pET-A cut by the same enzymes, generating pET-GFP.

The CAT-encoding gene was amplified using BL21(DE3)RIPL (Stratagene, La Jolla, Calif., USA) plasmid DNA (PCR primers, sense: 5′-AAAAAAGATATCGAG AAAAAAATCACTGGATATACCACCG-3′ (SEQ ID NO:5) and anti-sense: 5′-AAAAAAGCTAGCCGCCCCGCCCTGCCACTC-3′ (SEQ ID NO:6)), digested with EcoRV and NheI and ligated with pET-GFP to yield pET-CAT-GFP. Sequencing of pET-CAT-GFP indicated a deletion of the G in the NdeI site (lowercase g), resulting in a frameshift in the reading frame. To restore the reading frame, pET-CAT-GFP was digested with SpeI and protruding ends were endfilled and religated to yield the correct pETc-CAT-GFP. The plasmid pETc-CAT-GFP was used to express CAT-GFP.

The GK-encoding gene was amplified from E. coli DH12S genomic DNA (PCR primers, sense: 5′-AAAAAACTTAAGACTGAAAAAAAATATATCGTTGC GCTCGACC-3′ (SEQ ID NO:7) and anti-sense: 5′-AAAAAAGCTAGCTTCG TCGTGTTCTTCCCACGCC-3′ (SEQ ID NO:8)). The PCR products were digested by AflII and NheI (New England Biolabs, Ipswich, Mass., USA) and ligated with pET-GFP to yield pET-GK-GFP. Here again, the frameshift was corrected as for pET-CAT-GFP to obtain pETc-GK-GFP, which was used to express GK-GFP.

pETc-CAT-GFP and pETc-GK-GFP were digested with NheI, end filled and religated to introduce a stop codon at the end of the CAT and GK open reading frames to generate pET-CAT and pET-GK, respectively, which were used to express CAT and GK.

### Protein Expression and Purification

E. coli strain BL21-(DE3)-RIPL was used to express Tus, Tus-GFP, CAT, CAT-GFP, GK, GK-GFP, and GFP proteins. In this strain, the expression of the T7 RNA polymerase required to initiate the transcription of the inserts is controlled by the Lac promoter that is repressed in the presence of glucose. This strain is deficient in the Lon and OmpT proteases and contains extra copies of genes coding for tRNAs (RIPL) that may limit translation of heterologous proteins. Protein expression was not induced with IPTG.

An auto-inducible media was prepared according to the Studier protocol (ZY, MgSO₄, 1000× Trace Metal Mix, 20xNPS, 50×5052) (Studier Prot. Exp. Pur. 41:207-234, 2005) with the following modifications: sodium molybdate was replaced by ammonium molybdate, ZnSO₄ by ZnCl₂, cobalt chloride by cobalt sulphate, copper chloride by copper sulphate, Na₂SeO₃(3H₂0) by Na₂SeO₄, and N—Z Amine by peptone. GFP proteins were also expressed in commercial Overnight Express Instant Medium (Novagen, San Diego, Calif., USA).

Bacteria cultures were started from single colonies of overnight transformants plated on LB agar supplemented with 100 μg/ml ampicillin and 50 μg/ml chloramphenicol. Cell cultures (250 mls) where first incubated at 37° C. in 1 L flasks. Bacteria expressing GFP and GFP fusion proteins were transferred at 16° C. when they entered the stationary phase of growth at OD=6.6 (Overnight Express Instant Medium) and OD=11(Studier medium) to allow the proper folding of GFP. Cells were grown at 16° C. for 2 to 3 days until bacterial pellet (from centrifuged culture aliquots) showed bright fluorescence. Cells were harvested 48 hours after cells entered the stationary phase at OD=6.6 (Studier Media).

Cells were centrifuged at 8,000 rpm for 10 minutes at 4° C. in a Beckman Coulter (Fullerton, Calif., USA) Avanti J-20XP centrifuge and re-suspended in ice-cold lysis buffer (50 mM Na₂PO₄ [pH 7.8], 300 mM NaCl, 2 mM (3-mercaptoethanol) at 7 ml/g of cells. E. coli cells were lysed by two to three passes at 12,000 p.s.i. in a cooled French Pressure cell press. The lysate was centrifuged at 18,000 rpm for 40 minutes at 4° C. in a JA-20 rotor in a Beckman Coulter Avanti J-20xP centrifuge to eliminate cells debris. Cleared lysate was frozen in liquid nitrogen and stored at −80° C. until purification.

Proteins were purified using the Ni-charged resin Profinity IMAC (Bio-Rad, Hercules, Calif., USA). Briefly, 500 ml of resin was pre-equilibrated in lysis buffer prior to being added to the cleared lysate. His₆-Tagged proteins were allowed to bind nickel beads for 1 hour at 4° C. with rocking. The beads-containing lysate was next transferred into a standard filtered column and beads were allowed to settle to the bottom. The flow through (i.e., lysate minus beads) was passed twice through the column. Ni-charged beads were then washed 3 times with 1 ml and one time with 15 ml of lysis buffer supplemented with 10 mM imidazole. Retained proteins were eluted from the beads in lysis buffer supplemented with 200 mM imidazole. Elution fractions containing the proteins were pooled and proteins were precipitated by the addition of 0.5 g/ml (NH₄)₂SO₄ followed by one hour incubation at 4° C. under gentle shaking. The solution was then centrifuged at 18,000 rpm for 40 minutes at 4° C. The pellet obtained was resuspended in 1 ml of Buffer A (50 mM Na₂PO₄ [pH 7.8], 2 mM (β-mercaptoethanol) and was frozen in liquid nitrogen and stored at −80° C. Protein concentrations were determined by standard Bradford assay. Protein purity was assessed by NEXT-GEL SDS-PAGE (Amresco, Solon, Ohio, USA) and band quantification using the image analysis software ImageJ (see the website at rsbweb.nih.gov/ij/).

### DNA Ligands

Ter oligonucleotides were obtained from SIGMA-ALDRICH (St. Louis, Mo., USA), diluted in 10 mM Tris-HCl [pH 8], 1 mM EDTA (TE) supplemented with 50 mM KCl. Sequences are presented below. DNA ligands were prepared by heating at 75° C. for 5 minutes, followed by slow cooling of complementary pairs of oligonucleotides These DNA ligands correspond to previously described sequences (underlined) with the exception that they have each been extended with a GC rich dsDNA region in order to obtain Tm values >70° C. for each of them (Mulcair et al., Cell 125:1309-19, 2006).

The Ter oligonucleotide corresponds to the wild type sequence of Ter. Ter-AG is the Ter sequence lacking 2 nucleotides at the 3′-end of the strand containing the C₆ and corresponds to the Δ2p-rTerB in Mulcair et al. (Cell 125:1309-19, 2006). Ter-AAG is the Ter sequence lacking 3 nucleotides at the 3′-end of the strand containing the C₆ and corresponds to the Δ3p-rTerB in Mulcair et al. (Cell 125:1309-19, 2006). The TT-lock is the Ter variant with a 5-nucleotide overhang responsible for the locking of the Tus protein onto Ter by allowing the C₆ to flip out and bind the cytosine-binding pocket of Tus, resulting in a very strong bond (K_{D} in the picomolar range). This sequence corresponds to the Δ5n-rTerB oligonucleotide in Mulcair et al. (Cell 125:1309-19, 2006).

TerB
(SEQ ID NO: 9)
5′- CTTTAGTTACAACATACTTATCCCCGCCCCC
(SEQ ID NO: 10)
GAAATCAATGTTGTATGAATAGGGGCGGGGG
Ter-AG
(SEQ ID NO: 11)
5′- CTTTAGTTACAACATACTTATCACCCCGCCCCC
(SEQ ID NO: 12)
AATCAATGTTGTATGAATAGTGGGGCGGGGG
Ter-AAG
(SEQ ID NO: 13)
5′- CTTTAGTTACAACATACTTATCACCCCGCCCCC
(SEQ ID NO: 14)
ATCAATGTTGTATGAATAGTGGGGCGGGGG
TT-lock
(SEQ ID NO: 15)
5′- CCCCCGCCCCCAATACTTTAGTTACAACATACTTAT
(SEQ ID NO: 16)
GGGGGCGGGGGTTATGAAATCAATGTTGTAT

K_{D} (nM) values for the DNA ligands in 250 mM KCl—TerB: 1.4; Ter-AG: 16.5; Ter-AAG: 113; and TT-lock: 0.4.

### Determination of Thermal Aggregation Profiles and T_{agg}

Samples (6 or 10 μl) of Tus, Tus-GFP, CAT, CAT-GFP, GK, GK-GFP, and GFP, alone or as mixtures, were incubated in a thermocycler (Mycycler, BioRad, Hercules, Calif., USA) set on algorithm measurement for 15 μl sample volumes for 5 or 30 minutes along a temperature gradient. Sample Protein concentrations were typically between 10-14 μM in either Buffer A or in Buffer B (buffer A+10% v/v glycerol) in the case of Tus, Tus-GFP and GFP. After incubation, reactions were stopped by transferring the samples to ice for 10 minutes prior to centrifugation at 18,000 r.p.m. for 20 minutes at 4° C. in a Beckman Coulter (Fullerton, Calif., USA) centrifuge (rotor: F12×8.2). The supernatants (3 or 5 μl) were then analyzed by 10% NEXT-GEL SDS-PAGE (Amresco, Solon, Ohio, USA). The gels were illuminated on a transilluminator at 365 nm followed by Coomassie blue staining. Coomassie-stained protein bands corresponding to F_{fold} were integrated using ImageJ (see the website at rsbweb.nih.gov/ij/) and plotted against the temperature.

### S Method

Protein samples (6 or 10 μl) were incubated along a temperature gradient in a thermocycler for 5 minutes or 30 minutes for the determination of T_{agg}, or at a constant temperature and increasing times to determine k_{agg}. After heat treatment and centrifugation as described above, 3 or 5 μl of supernatant were transferred to a black 96-well plate (Nunclon, Nunc, Rochester, N.Y., USA), diluted with 47 or 50 μl respectively of Buffer A or Buffer B and the fluorescent F_{fold} was determined with a fluorescence plate reader (Victor V Wallace, Perkin-Elmer, Melbourne VIC, Australia). The excitation and emission filters were set at 355 nm and 535 nm respectively, with 40 nm band-width. Data were normalized against the fluorescence of an untreated sample.

To evaluate the effect of additives, Tus-GFP (13 μM) or GFP (control, 12 μM) in Buffer B were mixed with equal volumes of different additives in water. To determine the effect of DNA ligands, reaction samples containing 5.4 μl of Tus-GFP (11 μM in buffer B+272.2 mM KCl) and 0.6 μl of DNA ligand (100 μM in TE+50 mM KCl, pH 8) or TE+50 mM KCl, pH 8 for the control were incubated 10 minutes at 25° C. to allow complex formation prior to the heat denaturation step.

To determine the effect of ligands, CAT-GFP reaction premix contained 9.2 μl of CAT-GFP (71 μM in Buffer A), 51.6 μl of Buffer A and 4.2 μl of either 50 mg/ml chloramphenicol in ethanol or 50 mg/ml ampicillin in water. GK-GFP reaction premix contained 9.3 μl of GK-GFP (70 μM), 49.20 of Buffer A and 6.5 μl of either Buffer A+10 mM glycerol or Buffer A+10 mM glucose. Reaction volume was 10 μl and 5 μl of the soluble fraction was analysed by plate reader after centrifugation.

### EMSA

A modified version of an EMSA was used as an alternate method to confirm the T_{agg} of Tus-GFP-TerB complex obtained with the S method under the same conditions (FIG. 4). Briefly, equal volumes of Tus-GFP (˜80 μM in Buffer B) and TerB (100 μM in TE+50 mM NaCl, pH 8) were mixed, diluted 5 times in Buffer B and incubated at 25° C. for 10 minutes. The samples (6 μl) were heated in a thermocycler at the specified temperatures for 5 minutes, followed by 10 minutes on ice. Treated samples were loaded (4 μl) onto a 1% TBE-agarose gel where the F_{fold} of complexes were separated from the F_{agg} at 80 V for 20 minutes. Here, the binding of TerB to the Tus-GFP induces a shift in electrophoretic mobility of the complex towards the anode, whereas, unbound Tus-GFP (i.e., aggregated) stays in the wells. GFP fluorescence was detected at 365 nm and integrated with Image J (see the website at rsbweb.nih.gov/ij/) to determine the F_{fold} of Tus-GFP-TerB which showed increased mobility towards the anode.

### Data Fitting

To determine the T_{agg} at which 50% of proteins were aggregated, the thermal aggregation profile data were fit to the following sigmoid function:

F_{fold}=1−(1/(1+e^{(Tagg-T/c}))

where F_{fold} is the normalized fluorescence intensity at temperature T, and c is the Hill slope factor.

In the presence of TerB, the change in aggregation transition temperature ΔT_{agg} could be calculated as follows:

ΔT_{agg}=T_{agg(Tus-GFP-TerB)}−T_{agg(Tus)}

The k_{agg} (s⁻¹) measured the loss of fluorescence of the soluble fraction of proteins over time. The k_{agg} values were determined by the exponential fit of normalized F_{fold} as follows:

F_{fold}=e^{(kagg)t}

where t is the time in seconds.

### Results

### Fluorescent Proteins as a Reporter of Protein Stability/Aggregation

A fast and simple in vitro system using a fluorescent protein (e.g., GFP) as a reporter system to quantify the stability of a protein of interest and, optionally, its ligand-associated stabilization was designed.

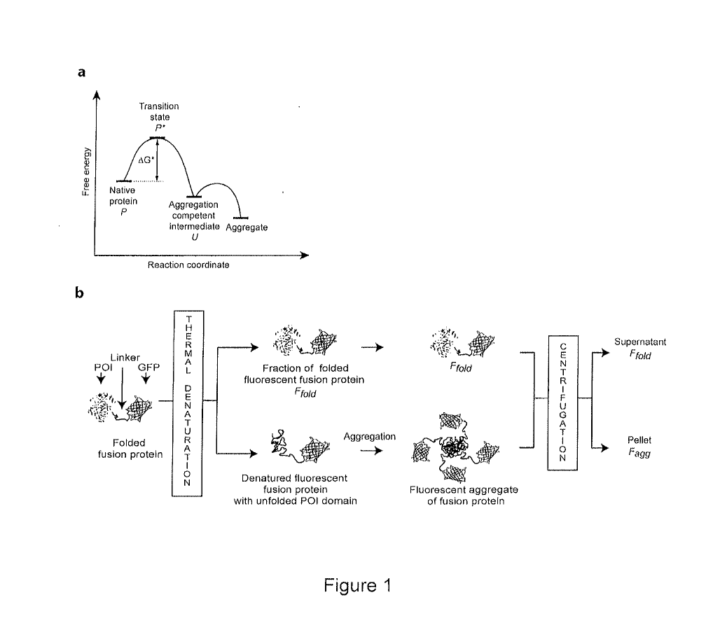

The in vitro system takes advantage of the fact that most folded proteins, when subjected to thermal denaturation, follow an unfolding pathway leading to irreversible protein aggregation as illustrated by the reaction coordinate diagram shown in FIG. 1a (see, Chi et al., Pharmaceut. Res. 20:1325-36, 2003).

It was rationalized that if GFP were to be used as a probe for protein unfolding and aggregation, then the unfolding of the protein of interest and the GFP domains in the fusion protein should be uncoupled (i.e., independent unfolding) to avoid influencing each other's unfolding kinetics (FIG. 1b). If the aggregation process has been completed, then the measurement of the residual population of folded proteins (non-aggregated) could be determined by measuring the fraction of protein that remains soluble (F_{fold}) after heat treatment. Consequently, the thermal stability of the protein of interest could be directly obtained through the measurement of the fluorescent F_{fold} of the GFP fusion protein after heat denaturation. In this case, the apparent aggregation rate constant (k_{agg}) reflects the unfolding kinetics of the protein of interest as the rate-limiting step is the unfolding process. As a result, the full range of physical and chemical conditions where GFP is stable and fluorescent can be used to monitor the aggregation properties of a less stable protein of interest (FIG. 1b).

As described herein, GFP-Basta is a sensitive method capable of quantitatively determining the stability of a protein of interest in the presence of other proteins. It requires neither special equipment nor extensive purification steps. GFP-Basta can accurately measure buffer and ligand-induced stabilization effects. GFP-Basta is easily amenable to various formats and its simplicity and speed offers an excellent strategy for the high-throughput determination of protein stability.

### Design of the Model Fusion Proteins

The thermal denaturation of well characterized proteins such as the monomeric DNA-binding protein Tus (Kamada et al., Nature 383:598-603, 1996), the trimeric chloramphenicol acetyl transferase (Panchenko et al., Biotechnol. Bioeng. 94:921-30, 2006) and the tetrameric glycerol kinase (Thorner et al., J. Biol. Chem. 248:3922-32, 1973; Koga et al., FEBS J. 275:2632-43, 2008) and their GFP-fusions were studied. Stabilization effects of various additives and ligands were also investigated.

The fusion constructs consisted of an N-terminal His₆-POI domain (including Tus, CAT and GK) followed by a minimal LGSGGH (SEQ ID NO:1) linker sequence and a C-terminal GFP. The linker was first used for the construction of a fully functional Tus-GFP fusion protein (Dandah et al., Chem. Commun. 3050-52, 2009). Tus binds to 21 by TerA-J sequences (Kamada et al., Nature 383:598-603, 1996; Mulcair et al., Cell 125:1309-19, 2006; Neylon et al., Microbiol. Mol. Biol. Rev. 69:501-26, 2005) and the association and dissociation rate constants of complex formation can be altered by mutating the Ter sequence, providing a useful tool to evaluate the effect of ligand affinity on Tus stability using GFP-Basta. The GFP was chosen due to its high excitability in the UV and its extreme stability in various conditions. The limits of GFP-Basta are therefore connected to the stability of GFP in the various tested conditions. The addition of CAT and GK demonstrate the universality of the system for other proteins and the identification of their ligands.

### Principle and Validation of the GFP Reporter System

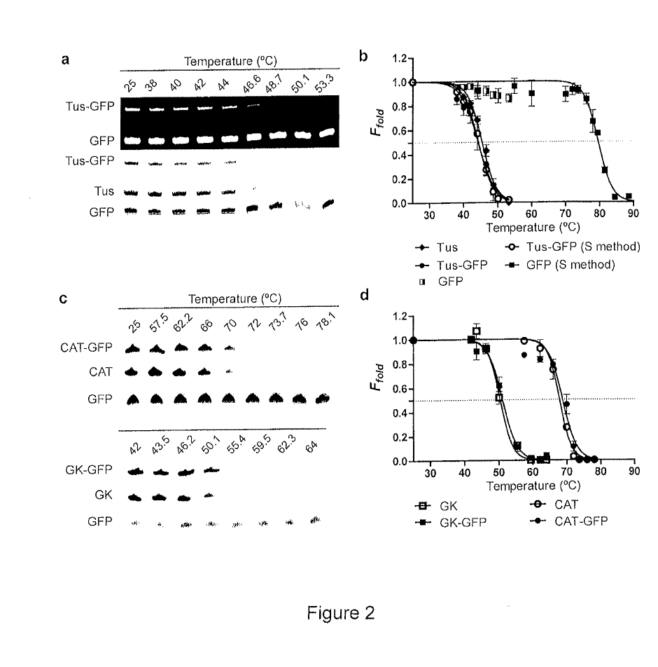

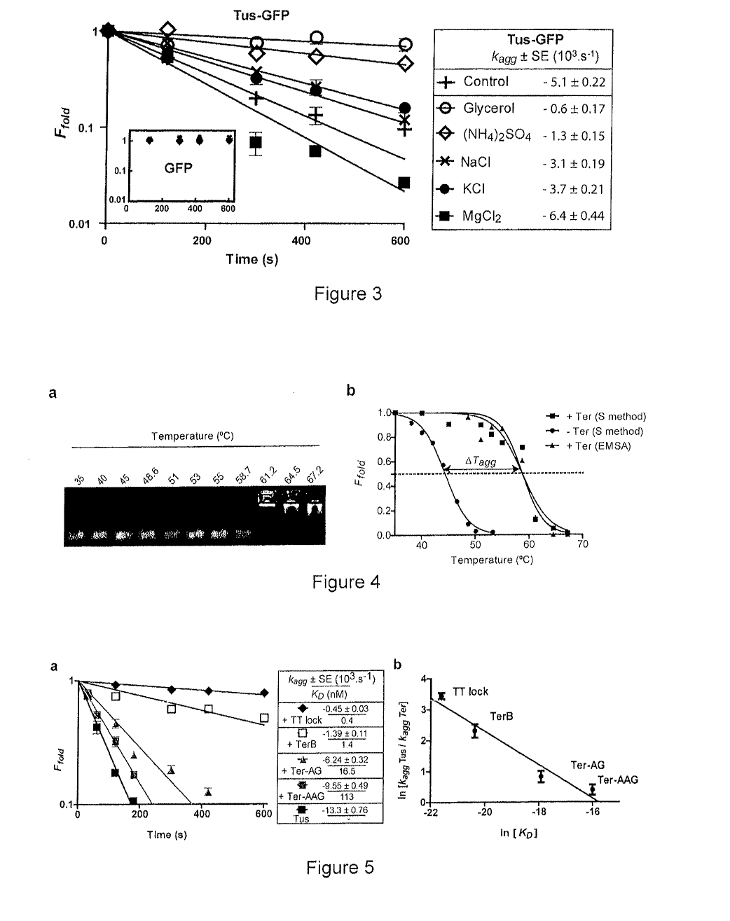

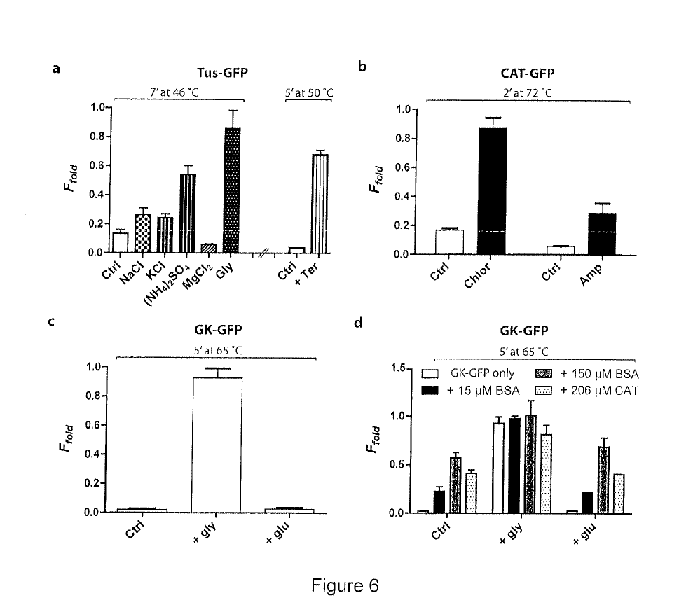

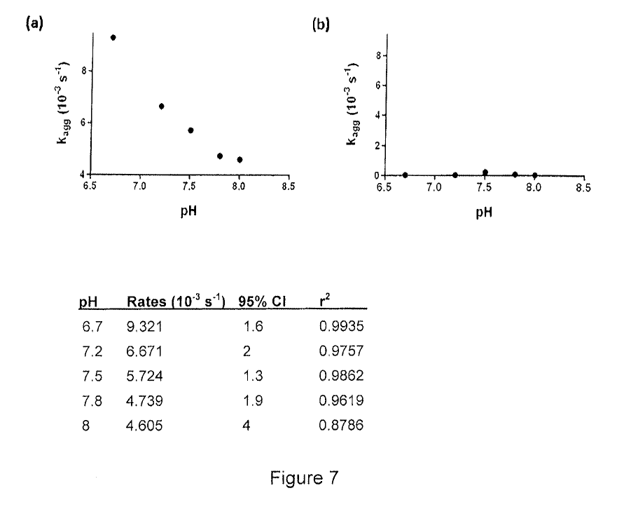

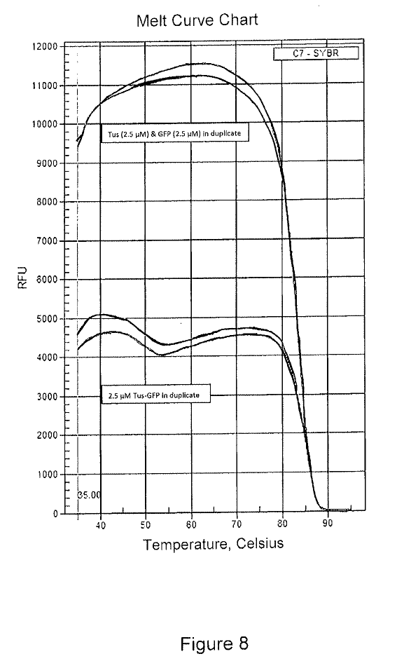

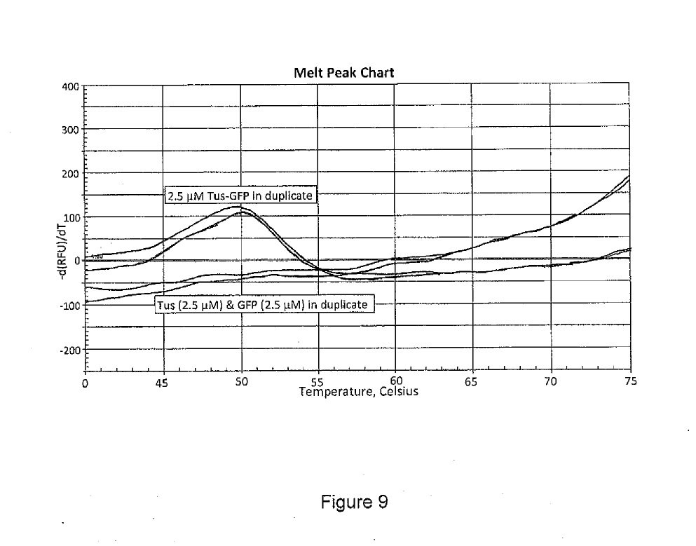

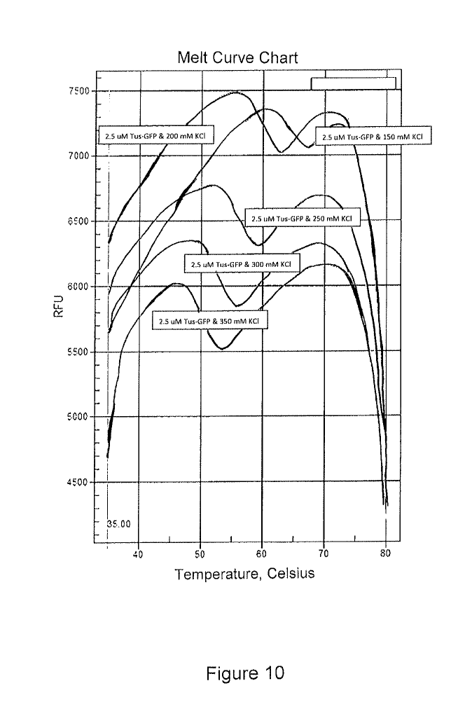

To show that the POI and GFP domains unfold independently, the respective stabilities of Tus, Tus-GFP and GFP were compared by incubating the proteins for 5 minutes at temperatures ranging from 25 to 53.3° C., followed by a cooling and centrifugation step to remove protein aggregates. F_{fold} was then determined by SDS-PAGE. For this experiment, equal amounts of the three proteins were mixed to avoid variations in buffer composition and protein concentrations. The T_{agg} values (temperature at which 50% of proteins are aggregated) from the thermal aggregation profiles for Tus and Tus-GFP were 45.4 and 44.2° C., respectively (FIGS. 2a and 2b). This demonstrated that the unfolding of the POI leading to its aggregation was unaltered in the GFP-fusion protein, and that no substantial effect was induced by the GFP domain.

The same was observed for CAT, GK and their GFP fusions (FIGS. 2c and 2d). Due to the oligomeric quaternary structure of these proteins, experiments were performed separately to avoid the formation of heterogeneous protein complexes. Here again, the T_{agg} values of CAT and GK were in agreement with their respective GFP fusions (FIGS. 2c and 2d). The data indicate that the aggregation rate constants (k_{agg}), and therefore all preceding unfolding processes, must be essentially identical for the POI-GFP and POI.

As expected, GFP was not affected in this temperature range (Ishii et al., Appl Biochem. Biotech. 137:555-71, 2007; Ishii et al., Int. J. Pharm. 337:109-17, 2007). The T_{agg} of GFP was determined to be 79.6° C. by measuring its residual fluorescence after heat denaturation and centrifugation at a higher temperature range using a fluorescence plate reader (the S method) as readout. The T_{agg} of Tus-GFP was also reproduced using the S method (44.3° C.; FIG. 2b). Furthermore, the total fluorescence including F_{fold} and F_{agg} remained unaltered if heat denaturation occurred at temperatures below ˜75° C. for 5 minutes, meaning that GFP was still folded in the fusion protein aggregates and that aggregation of the POI portion did not trigger the unfolding of GFP. The aggregation of POI-GFP is therefore the result of the unfolding of its most unstable POI domain. These results validate the hypothesis and demonstrate that the different linkers are long enough to uncouple the unfolding of POI and GFP in the fusion protein

### Isothermal Aggregation and Evaluation of Additives

To evaluate the kinetic parameters of the system, a 96-well plate format was designed that enabled measurement of the residual F_{fold} of Tus-GFP over time. Isothermal aggregation reactions were monitored at 46° C. to quantify the effect of stabilizing or destabilizing salts and additives on the k_{agg} of Tus-GFP (FIG. 3). Here, an increase in protein stability due to an additive is reflected by a decrease in k_{agg} compared to the Tus-GFP control sample (without additive). As each additive or salt might influence the stability of the Tus or GFP portion of the fusion protein, it is important to use a GFP control to show that the additive does not affect the fluorescence or stability of GFP.

FIG. 3 illustrates a few examples of additives commonly used in storage buffers to improve protein stability. These additives did not affect the stability or fluorescence of GFP (FIG. 3). Glycerol was found to have the largest stabilization effect on Tus-GFP. It is therefore possible to quickly screen for optimal protein storage conditions using GFP-Basta.

### TerB Induced Stabilization of Tus

Protein stability is generally increased by ligand binding (Jelesarov and Bosshard, J. Mol. Recognit. 12:3-18, 1999). Tus is a DNA binding protein that binds to 21 by DNA sequences called Ter (Kamada et al., Nature 383:598-603, 1996; Mulcair et al., Cell 125:1309-19, 2006; Neylon et al., Microbiol. Mol. Biol. Rev. 69:501-26, 2005). It was expected that the tight binding of TerB to Tus-GFP should therefore induce a strong ligand-induced stabilization effect resulting in a large shift in T_{agg} (ΔT_{agg}). The T_{agg} of the Tus-GFP-TerB complex was first determined by a modified electrophoretic mobility shift assay (EMSA; FIG. 4a) (Dandah et al., Chem. Commun. 3050-52, 2009).

Tus-GFP and TerB were mixed in equimolecular quantities in low salt conditions (K_{D}<pM) and treated at room temperature for 10 minutes to allow complex formation prior to being heat-treated at temperatures ranging from 35 to 67.2° C. Here, no centrifugation step was required as the Tus-GFP aggregates were retained in the wells of the agarose gel due to their low mobility, and Ter-bound Tus-GFP proteins corresponding to F_{fold} migrated more rapidly due to their increased net negative charge. The bands corresponding to F_{fold} were integrated and revealed a T_{agg} of 58.7° C. corresponding to an increase in thermostability of 14.4° C.

Heat induced aggregation curves of Tus-GFP and Tus-GFP-TerB were also obtained in the same conditions and compared using the fluorescence plate reader after a centrifugation step (FIG. 4b; the S method). The T_{agg} of the Tus-GFP-TerB complexes, obtained with the two different methods, were essentially the same confirming that F_{fold} consists mainly of folded and active proteins.

### Relationship Between Ligand Affinity and Aggregation Rates of Tus-Ter Complex

Using the Tus-Ter model system, the ligand induced stabilization on Tus-GFP of various well-characterized DNA ligands by isothermal aggregation reactions using the S method was investigated. The dissociation constants (K_{D}), for various Tus-Ter complexes (TerB, Ter-AG, Ter-AAG and TT-lock) were previously determined by surface plasmon resonance (SPR; Mulcair et al., Cell 125:1309-19, 2006).

Here, the relationship between K_{D} and k_{agg} in conditions where the Tus-GFP-Ter complexes were at concentrations at least ˜100 fold above their respective K_{D} were determined, to ensure that at least 99% of proteins were in their bound form. The k_{agg} values of the complexes were determined at 50° C. in 250 mM KCl, where unbound Tus-GFP proteins aggregate very quickly. As expected, the k_{agg} values of the complexes increased with increasing K_{D} values (FIG. 5a). It was rationalized that ligand-induced stabilization effects due to the gain of inter- and intramolecular interactions could simply be extracted by dividing the k_{agg} of Tus by the k_{agg} of the complex, and this value should correlate with the K_{D} of the interaction. Indeed, a linear correlation was obtained between ln(K_{D}) from published SPR data (Mulcair et al., Cell 125:1309-19, 2006) and the ln(k_{agg(Tus}/k_{agg(Tus-Ter)}) obtained by GFP-Basta (FIG. 5b).

Others have studied the stabilization effect of glycerol on the irreversible thermal denaturation of creatine kinase using the activated-complex theory (see, e.g., Meng et al., Biophys. J. 87:2247-54, 2004). Here, this theory was used to demonstrate the relationship between ln K_{D} and the ln(k_{agg(Tus)}/k_{agg(Tus-Ter)}) seen in FIG. 5b. The activated complex is an intermediate transition state between reactants and products. The activated-complex theory postulates the existence of an equilibrium between reactants (P) and the activated complex (P*). In this case, the kinetic scheme of irreversible denaturation and aggregation of a protein is expressed as:

$$P {⇔}^{{K}^{*}} {P}^{*} \to U {\to }^{kagg} Aggregate$$

It was rationalized that the fraction of unfolded proteins after heat denaturation would be driven into an irreversible aggregation pathway. The extent of aggregation should therefore reflect the proportion of unfolded proteins. In this case, the apparent aggregation rate constant k_{agg} is related to the change in free energy of activation ΔG* and can be expressed in accordance with the activated-complex theory (Meng et al., Biophys. J. 87:2247-54, 2004) as:

k_{agg}=(k_{B}T/h)e^{(-ΔG*/RT)}

which can be transformed to:

ΔG*=−RT ln k_{agg}(h/k_{B}T)

The difference in change of free energy of activation (ΔΔG*) between Tus-GFP (ΔG*_{Tus}) and Tus-GFP-ligand (ΔG*_{Tus-Ter}) can be obtained with the following expression:

ΔΔG*=ΔG*_{Tus}−ΔG*_{Tus-Ter}=−RT ln(k_{agg(Tus)}/k_{agg(Tus-Ter)}) (a)

ΔG* is connected with the equilibrium constant by the relationship ΔG*=−RT ln K*. This term can be replaced in equation (a) giving:

ln K*_{(Tus)}−ln K*_{(TUs-Ter)}=ln(k_{agg(Tus)}/k_{agg(Tus-Ter)}) (b)

In the situation where most of Tus is in complex with its ligand, the term ln K*_{Tus-Ter} can be represented as the sum of ln K*_{(Tus)} and the ligand-induced stabilization of Tus given by ln K*_{(ligand effect)}. The expression (b) can therefore be simplified as:

−ln K*_{(lignad effect)}=ln(k_{agg(Tus)}/k_{agg(Tus-Twe)}) (c)

ln K*_{(ligand effect)} is proportional to the ΔΔG* induced only by ligand binding and should therefore be proportional to ln K_{D} of the Tus-ligand complex. To test this, lnK*_{(ligand effect)} was replaced with the term ln K_{D} in the relationship (c) and a linear correlation was obtained between ln(k_{agg(Tus)}/k_{agg(Tus-Ter)}) and ln K_{D} (FIG. 5b). GFP-Basta can therefore be used to accurately estimate the K_{D} values of Ter ligands.

### Rapid Throughput Screening

The S method was then further developed to evaluate the effect of ligand binding and additives in a rapid throughput format for the three test proteins using replicates of single timepoints (FIG. 6a-6c). The assay showed high specificity for Tus-GFP and GK-GFP for their specific ligands, whereas CAT-GFP due to its extreme thermostability (Panchenko et al., Biotechnol. Bioeng. 94:921-30, 2006) was borderline. Indeed, the temperature used to test for chloramphenicol binding had to be kept quite low and for a short time as GFP would have been destabilized in more stringent conditions. As expected the presence of chloramphenicol proved to be stabilizing (FIG. 6b). The effect of ampicillin, which is not a ligand of CAT, was also tested, and a moderate stabilization of CAT-GFP was observed. The presence of 6.4% ethanol present in one of the control experiment had almost the same stabilizing effect, which suggests that CAT could be stabilized by non-specific hydrophobic interactions. This non-specific stabilization could also be attributed to the low stringency of the conditions.

Additionally, the stability of GK-GFP in presence of excess CAT or BSA concentrations ranging from 15-206 μM was tested(FIG. 6d). This experiment was setup to evaluate performing reactions in mixtures of proteins or to monitor the stabilization effect of another protein, which is an advantage over other thermal denaturation based techniques such as thermofluor. BSA and CAT were chosen because they are stable in the reaction conditions. The results show that at high concentrations of either CAT or BSA, GK-GFP was strongly stabilized but not to the extent seen in presence of its glycerol ligand. When BSA was present in only slight excess (15 μM), GK-GFP was much less stabilized. Nevertheless, addition of the glycerol ligand almost completely stabilized GK-GFP under these conditions. These results show that in presence of moderately high concentrations of another protein, it is still possible to use the assay to identify the effects of ligands or additives.

### Buffer Effect on Tus-GFP

In order to characterize the sensitivity of Tus to pH, Tus-GFP aggregation rates were monitored in phosphate buffers (50 mM) with a pH of 6.7, 7.2, 7.8, and 8 at 46° C. (FIG. 7).

Tus-GFP stability was found to be pH dependant with a slower aggregation rate at higher pH values. It is likely that the pattern observed in FIG. 7a corresponds to the first half of a parabola that illustrates the pH dependence of Tus-GFP, with pH 7.8 and 8 being the optimal pH for the protein.

### GFP-Basta Thermoscreen

In order to characterize GFP-Basta in the absence of separating the fusion protein into soluble and insoluble fractions following exposure to a test condition, a thermoscreen procedure was developed, with GFP acting as a probe to monitor protein unfolding and/or aggregation of Tus (an exemplary POI) through the GFP fluorescence quenching occurring upon Tus unfolding and/or aggregation.

The incremental heating of Tus-GFP results in a loss of fluorescence occurring at the transition temperature (T_{agg}) at which the fusion protein unfolds and aggregates, leading to fluorescence quenching.

First, it was confirmed that a T_{agg} can only be obtained with a Tus-GFP fusion protein, as opposed to a mixture of the individual Tus and GFP proteins (FIGS. 8 and 9). Here, two different samples were analysed. The first sample contained 2.5 μM Tus-GFP fusion protein. The second sample contained a mixture of 2.5 μM Tus protein and 2.5 μM GFP protein. These samples were loaded on a PCR plate that was then heated in a real-time thermocycler (BioRad, Hercules, Calif., USA) using the melting curve protocol according to the manufacturer's instructions, modified as follows: the start temperature was set at 35° C. and the end temperature was set at 80° C., with 10 seconds dwell time every 0.5° C. (40 minute protocol) (FIG. 8). These parameters can be altered depending on the T_{agg} of the protein of interest. The T_{agg} is determined using the instrument's software by plotting dRFU/dT against temperature, and is represented as a peak. The peak maximum represents the T_{agg}.

Under these conditions, a T_{agg} of 50° C. could only be detected from the traces obtained with the Tus-GFP fusion protein (FIG. 9). No detectable T_{agg} (no peak) could be detected with the mixture of Tus and GFP proteins (FIG. 9), demonstrating the necessity of using POI-GFP fusion proteins to obtain a T_{agg}.

The binding of Ter ligands to Tus in the Tus-GFP fusion protein result in a shift in T_{agg} (ΔT_{agg}). For the analysis, 60 μl of protein-ligand sample in a concentration range of approximately 2.5 μM provides a reasonable signal-to-noise ratio in a thermocycler with real-time capability. Melting curves are generated using the instrument's software to obtain the T_{agg} (FIG. 10).

Here, Ter oligonucleotides (Ter A-J and OriC; sequences presented below) were diluted to 7.5 μM in Buffer A without salt and mixed in a 96-well plate with one volume of Buffer A, three times final desired KCl concentrations (from 73.5 mM to 973.5 mM) and one volume of Tus-GFP at 7.5 μM in Buffer A without salt. The final reaction mixtures therefore contained 2.5 μM of Tus-GFP and Ter or oriC and varying concentrations of KCl ranging between 39.5 mM and 339.5 mM KCl. The plate was then heated in a real-time thermocycler (BioRad, Hercules, Calif., USA) using the melting curve protocol according to the manufacturer's instructions, modified as follows: the start temperature was set at 35° C. and the end temperature was set at 80° C., with 10 seconds dwell time every 0.5° C. (40 minute protocol). These parameters can be altered depending on the T_{agg} of the protein of interest. The T_{agg} is determined as the maximum of the derivative of the sigmoidal curve obtained by plotting the fluorescence signal against temperature.

Transformed traces indicated that T_{agg} varied from 51-65° C. as a result of decreasing KCl concentrations from 350-150 mM. The T_{agg} values so obtained were then plotted against KCl concentrations (Table I).

Ter A
(SEQ ID NO: 17)
5′ -AATTAGTATGTTGTAACTAAAGTGGGGGCGGGG
(SEQ ID NO: 18)
TTAATCATACAACATTGATTTCACCCCCGCCCC
Ter B
(SEQ ID NO: 19)
5′ -AATAAGTATGTTGTAACTAAAGTGGGGGCGGGG
(SEQ ID NO: 20)
TTATTCATACAACATTGATTTCACCCCCGCCCC
Ter C
(SEQ ID NO: 21)
5′ -ATATAGGATGTTGTAACTAATATGGGGGCGGGG
(SEQ ID NO: 22)
TATATCCTACAACATTGATTATACCCCCGCCCC
Ter D
(SEQ ID NO: 23)
5′ -CATTAGTATGTTGTAACTAAATGGGGGGCGGGG
(SEQ ID NO: 24)
GTAATCATACAACATTGATTTACCCCCCGCCCC
Ter E
(SEQ ID NO: 25)
5′ -TTAAAGTATGTTGTAACTAAGCAGGGGGCGGGG
(SEQ ID NO: 26)
AATTTCATACAACATTGATTCGTCCCCCGCCCC
Ter F
(SEQ ID NO: 27)
5′ -CCTTCGTATGTTGTAACGACGATGGGGGCGGGG
(SEQ ID NO: 28)
GGAAGCATACAACATTGCTGCTACCCCCGCCCC
Ter G
(SEQ ID NO: 29)
5′ -GTCAAGGATGTTGTAACTAACCAGGGGGCGGGG
(SEQ ID NO: 30)
CAGTTCCTACAACATTGATTGGTCCCCCGCCCC
Ter H
(SEQ ID NO: 31)
5′ -CGATCGTATGTTGTAACTATCTCGGGGGCGGGG
(SEQ ID NO: 32)
GCTAGCATACAACATTGATAGAGCCCCCGCCCC
Ter I
(SEQ ID NO: 33)
5′ -AACATGGAAGTTGTAACTAACCGGGGGGCGGGG
(SEQ ID NO: 34)
TTGTACCTTCAACATTGATTGGCCCCCCGCCCC
TerJ
(SEQ ID NO: 35)
5′ -ACGCAGTAAGTTGTAACTAATGCGGGGGCGGGG
(SEQ ID NO: 36)
TGCGTCATTCAACATTGATTACGCCCCCGCCCC
oriC
(SEQ ID NO: 37)
5′ -CCGGCTTTTAAGATCAACAACCTGGAAAGGATCA
(SEQ ID NO: 38)
GGCCGAAAATTCTAGTTGTTGGACCTTTCCTAGT

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

TABLE I
The effect of increasing KCl concentrations on the binding of ten different Ter
ligands (A-J) and a non-specific DNA (OriC) to Tus-GFP analysed simultaneously.
The T_{agg} for each condition is listed.

| | Ter | | | | | | | | | | | |
|---|---|---|---|---|---|---|---|---|---|---|---|---|
| [KCL mM] | A | B | C | D | E | F | G | H | I | J | OriC | -- |
| 14.9 | 72.3 | 72.5 | 72.6 | 72.4 | 72.2 | 70.0 | 74.7 | 72.9 | 74.4 | 72.4 | 59.1 | 44.9 |
| 39.4 | 72.2 | 72.4 | 72.2 | 72.1 | 71.3 | 67.7 | 74.5 | 71.4 | 72.7 | 70.7 | 55.9 | 44.9 |
| 89.4 | 70.2 | 71.0 | 69.9 | 70.2 | 69.5 | 63.9 | 69.0 | 68.0 | 68.6 | 67.0 | 52.0 | 45.4 |
| 139.4 | 66.0 | 66.3 | 66.2 | 66.2 | 65.1 | 58.6 | 64.9 | 63.4 | 63.2 | 61.8 | 48.0 | 45.2 |
| 189.4 | 61.7 | 63.0 | 61.4 | 61.5 | 60.5 | 52.7 | 61.1 | 59.0 | 58.4 | 57.4 | 46.8 | 45.6 |
| 239.4 | 58.7 | 58.5 | 58.0 | 58.0 | 56.4 | 48.3 | 57.5 | 54.4 | 54.1 | 53.2 | 45.5 | 45.5 |
| 289.4 | 54.9 | 55.7 | 54.4 | 55.2 | 53.1 | 46.5 | 53.7 | 51.2 | 50.7 | 49.1 | 45.4 | 45.8 |
| 339.4 | 52.6 | 52.5 | 52.0 | 51.5 | 50.6 | 45.5 | 51.4 | 48.2 | 48.2 | 46.6 | 45.9 | 46.1 |

The (--) represents the negative control: Tus-GFP in absence of DNA.

1. A method for assessing protein solubility, the method comprising the steps of:
a) exposing a fusion protein to a test condition, said fusion protein comprising a protein of interest, a linker and a fluorescent marker protein, wherein said marker protein does not affect the solubility of said protein of interest;
b) separating said fusion protein into soluble and insoluble fractions, wherein said soluble fraction comprises soluble fusion protein and said insoluble fraction comprises aggregates of said fusion protein; and
c) measuring residual fluorescence of said fusion protein in said soluble fraction as an indicator of protein solubility.

2. The method of claim 1, further comprising the step of producing and/or purifying said fusion protein prior to exposing said fusion protein to said test condition.

3. The method of claim 2, wherein the fusion protein is produced by:
expressing said fusion protein in an expression system, wherein said expression system comprises a nucleic acid molecule encoding said fusion protein and a promoter active in said expression system operably linked to said nucleic acid molecule; and
extracting a protein sample from said expression system, wherein said protein sample comprises said fusion protein.

4. The method of claim 3, wherein said expression system comprises an expression construct, wherein said nucleic acid molecule is operably linked to one or more regulatory sequences in said expression construct and said promoter is active in a host cell, and said fusion protein is expressed in said host cell.

5. The method of claim 3, wherein said expression system comprises an in vitro transcription/translation system.

6. The method of claim 1, wherein producing a fusion protein comprises joining said protein of interest via said linker to said fluorescent marker protein.

7. The method of claim 1, wherein said fluorescent marker protein is C-terminal to said protein of interest.

8. The method of claim 1, wherein said fluorescent marker protein is N-terminal to said protein of interest.

9. The method of claim 1, wherein said linker comprises 5 to 50 amino acids.

10. The method of claim 1, wherein said linker comprises the amino acid sequence LGSGGH (SEQ ID NO:1).

11. The method of claim 1, wherein said test condition is selected from the group consisting of a physical treatment, a chemical treatment and addition of one or more ligands.

12. The method of claim 11, wherein said physical treatment is selected from the group consisting of a change in temperature, a change in pH, a change in ionic strength, a change in salt concentration, and combinations thereof.

13. The method of claim 11, wherein said chemical treatment is selected from the group consisting of addition of an oxidizing agent, addition of a reducing agent, addition of a detergent, and combinations thereof.

14. The method of claim 11, wherein said one or more ligands is selected from the group consisting of a protein, a metal ion and a small molecule.

15. The method of claim 1, wherein exposing said fusion protein to said test condition occurs in a well of a microtiter plate.

16. The Method of claim 1, wherein separating said fusion protein into soluble and insoluble fractions comprises centrifugation of said fusion protein following exposure to said test condition.

17. The method of claim 1, wherein separating said fusion protein into soluble and insoluble fractions comprises spotting an aliquot of said fusion protein onto a selectively permeable matrix following exposure to said test condition.

18. The method of claim 17, wherein said selectively permeable matrix comprises an agarose gel or a polyacrylamide gel.

19. A method for assessing protein solubility, the method comprising the steps of:
a) expressing a fusion protein in an expression system, wherein said expression system comprises
(i) a nucleic acid molecule encoding said fusion protein, said fusion protein comprising a protein of interest, a linker and a fluorescent marker protein, wherein said marker protein does not affect the solubility of said protein of interest, and
(ii) a promoter active in said expression system operably linked to said nucleic acid molecule;
b) extracting a protein sample from said expression system, wherein said protein sample comprises said fusion protein;
c) exposing said fusion protein to a test condition;
d) separating said fusion protein into soluble and insoluble fractions, wherein said soluble fraction comprises soluble fusion protein and said insoluble fraction comprises aggregates of said fusion protein; and
e) measuring said residual fluorescence of said fusion protein in said soluble fraction as an indicator of protein solubility.

20. The method of claim 1, wherein assessing protein solubility comprises assessing protein stability.

21. The method of claim 20, wherein said protein of interest is a mutant protein comprising one or more amino acid substitutions, insertions or deletions, and wherein assessing protein stability comprises assessing the stability of said mutant protein.

22. The method of claim 20, wherein assessing protein stability comprises assessing changes in protein stability upon binding of a ligand.

23. The method of claim 1, further comprising screening potential inhibitors of protein aggregation, wherein exposing said fusion protein to said test condition includes exposing said fusion protein to a potential inhibitor of protein aggregation.

24. A method for assessing protein stability, the method comprising the steps of:
a) exposing a fusion protein to a test condition, said fusion protein comprising a protein of interest, a linker and a fluorescent marker protein, wherein said marker protein does not affect the stability of said protein of interest;
b) heating said fusion protein; and
c) monitoring fluorescence quenching of said fusion protein as an indicator of protein stability.

25. The method of claim 24, wherein said linker comprises 5 to 50 amino acids.

26. The method of claim 24, wherein said linker comprises the amino acid sequence LGSGGH (SEQ ID NO:1).

27. The method of claim 24, wherein said test condition is selected from the group consisting of a physical treatment, a chemical treatment and addition of one or more ligands.

28. The method of claim 27, wherein said physical treatment is selected from the group consisting of a change in temperature, a change in pH, a change in ionic strength, a change in salt concentration, and combinations thereof.

29. The method of claim 27, wherein said chemical treatment is selected from the group consisting of addition of an oxidizing agent, addition of a reducing agent, addition of a detergent, and combinations thereof.

30. The method of claim 27, wherein said one or more ligands is selected from the group consisting of a protein, a metal ion and a small molecule.

31. An isolated nucleic acid molecule, comprising a polynucleotide encoding a peptide linker in-frame with a polynucleotide encoding a fluorescent protein, and an internal cloning site into which a heterologous polynucleotide encoding a protein of interest can be inserted in-frame with said linker and said fluorescent protein coding sequences.

32. An isolated fusion protein, comprising an amino acid sequence of a protein of interest, a peptide linker amino acid sequence and an amino acid sequence of a fluorescent marker protein.

33. The fusion protein of claim 32, wherein said peptide linker amino acid sequence comprises LGSGGH (SEQ ID NO:1).

34. The fusion protein of claim 32, wherein said fluorescent marker protein is selected from the group consisting of green fluorescent protein, yellow fluorescent protein, blue fluorescent protein, red fluorescent protein, and orange fluorescent protein.

35. The fusion protein of claim 32, wherein said fluorescent marker protein is green

35. The fusion protein of claim 32, wherein said fluorescent marker protein is green fluorescent protein.

36. A kit for assessing protein solubility, said kit comprising an expression vector comprising a polynucleotide encoding a peptide linker in-frame with a polynucleotide encoding a fluorescent protein, and an internal cloning site into which a heterologous polynucleotide encoding a protein of interest can be inserted in-frame with said linker and said fluorescent protein coding sequences.

37. A kit for assessing protein solubility, said kit comprising one or more oligonucleotide primer pairs for introducing a promoter, a ribosomal binding site, and a linker for generating a fusion gene comprising a gene coding for a protein of interest joined in-frame with said fluorescent marker gene.
